(19) United States
(12) Patent Application Publication
(10) Pub. No.: US 2016/0346350 A1
(43) Pub. Date: Dec. 1, 2016

(54) COMPOSITIONS AND METHODS FOR TREATING OR PREVENTING A BONE CONDITION

(71) Applicant: THE REGENTS OF THE UNIVERSITY OF CALIFORNIA, Oakland, CA (US)

(72) Inventors: Cun-yu Wang, Los Angeles, CA (US); Bo Yu, Los Angeles, CA (US); Jia Chang, Ann Arbor, MI (US)

(21) Appl. No.: 15/114,800

(22) PCT Filed: Jan. 29, 2015
(86) PCT No.: PCT/US15/13591

§ 371 Date: Jul. 27, 2016

Related U.S. Application Data
(60) Provisional application No. 61/933,223, filed on Jan. 29, 2014.

(51) Int. Cl. A61K 38/17

(52) U.S. Cl. CPC A61K 38/1709

(57) ABSTRACT

The present invention discloses a composition for bone regeneration in a subject and method of using and making the same.

### CROSS-REFERENCE TO RELATED APPLICATIONS

This application claims the benefit of U.S. provisional application No. 61/933,223, filed Jan. 29, 2014, the teaching of which is incorporated herein in its entirety by reference.

### STATEMENT OF FEDERAL GOVERNMENT SUPPORT

This invention was made with Government support under Grant No. R01DE19412 awarded by the National Institute of Dental and Craniofacial Research (NIDCR) and the National Institutes of Health (NIH). The Government has certain rights in the invention.

### BACKGROUND OF THE INVENTION

The present invention is generally related to pharmaceutical compositions for treating or preventing bone condition such as osteoporosis or an inflammatory bone disorder.

Aging-related bone loss and osteoporosis affect millions of patients worldwide. Chronic inflammation associated with osteoporosis arthritis, and periodontitis promotes bone resorption and impairs bone formation.

Normal bone remodeling maintains constant bone mass by an orchestrated balance between the destruction of pre-existing bone by osteoclasts and rebuilding by osteoblasts^{1,2}. Osteoporosis brings significant changes to the skeletal system, characterized by structural alterations including reduction in trabecular bone volume, density and strength^{3,4}, as well as a shift in tissue microenvironment with increasing pro-inflammatory cytokine levels in bone marrow and the serum⁵⁻⁸. Advancing age is also a critical risk factor for osteoporosis which is the most common metabolic bone disease and a leading cause of morbidity and mortality in our aging population. It is estimated that bone fracture rates due to osteoporosis surpass the combined incidence of breast cancer, stroke, and heart attacks in postmenopausal women⁹⁻¹¹.

The canonical Wnt/beta-catenin signaling pathway has been found to play an important role in bone formation and skeletal development (e.g., Lyons, J P, et al., Exp Cell Res. 2004 Aug. 15; 298(2):369-87; Chang, J., et al., J Biol Chem. 2007 Oct. 19; 282(42):30938-48. Epub 2007 Aug. 24). However, it is unknown whether non-canonical Wnt4 play a role in osteoporosis and arthritis. The canonical Wnt proteins such as Wnt1 and Wnt10a may promote bone formation, but they might also increase the risk for cancer development. Although Wnt5a, a non-canonical Wnt family member protein, can promote osteoblast differentiation, it might also stimulate osteoclast formation, which could lead to bone loss. Therefore, these Wnt proteins might be not good therapeutic agents for preventing bone loss.

Therefore, there is a continuing need for additional agents and methods for treating, ameliorating or preventing a bone disorder.

The embodiments described below address the above-identified problems and needs.

### SUMMARY OF THE INVENTION

In one aspect of the present invention, it is provided a method for treating, ameliorating, or preventing a bone condition, comprising administering to a subject in need thereof a biologically active agent in an effective amount for promoting bone formation and inhibiting bone resorption independent of Wnt/b-catenin signaling to promote bone repair or regeneration in the subject.

In some embodiments of the method of invention, the biologically active agent is a Wnt4 protein.

In some embodiments of the method of invention, optionally in combination with any of the various embodiments of invention method herein, the Wnt4 protein is included in a pharmaceutical composition.

In some embodiments of the method of invention, optionally in combination with any of the various embodiments of invention method herein, administering comprising administering to the subject a gene construct encoding the Wnt4 protein.

In some embodiments of the method of invention, optionally in combination with any of the various embodiments of invention method herein, administering comprising transfecting a cell of the subject a gene construct encoding the Wnt4 protein.

In some embodiments of the method of invention, optionally in combination with any of the various embodiments of invention method herein, administering comprises administering to the subject an mRNA encoding the Wnt4 protein.

In some embodiments of the method of invention, optionally in combination with any of the various embodiments of invention method herein, the pharmaceutical composition is in a formulation for systemic delivery.

In some embodiments of the method of invention, optionally in combination with any of the various embodiments of invention method herein, the pharmaceutical composition is in a formulation for local delivery.

In some embodiments of the method of invention, optionally in combination with any of the various embodiments of invention method herein, the subject is a human being.

In some embodiments of the method of invention, optionally in combination with any of the various embodiments of invention method herein, the bone condition is one of osteoporosis, inflammatory bone diseases, periodontal diseases, and chronic diseases-associated bone loss. An example of the inflammatory bone disease is arthritis.

In a second aspect of the present invention, it is provided a composition for bone regeneration in a subject, comprising a biologically active agent in an effective amount for promoting bone formation and inhibiting bone resorption in the animal independent of Wnt/b-catenin signaling.

In some embodiments of the composition of invention, the biologically active agent is a Wnt4 protein.

In some embodiments of the composition of invention, optionally in combination with any of the various embodiments of invention composition herein, the Wnt4 protein is included in a pharmaceutical composition.

In some embodiments of the composition of invention, optionally in combination with any of the various embodiments of invention composition herein, the pharmaceutical composition is in a formulation for systemic delivery.

In some embodiments of the method of composition, optionally in combination with any of the various embodiments of invention composition herein, the composition comprises a gene construct encoding the Wnt4 protein.

In some embodiments of the method of composition, optionally in combination with any of the various embodiments of invention composition herein, the composition comprises an mRNA encoding the Wnt4 protein.

In some embodiments of the composition of invention, optionally in combination with any of the various embodiments of invention composition herein, the pharmaceutical composition is in a formulation for local delivery.

In some embodiments of the method of invention, optionally in combination with any of the various embodiments of invention composition herein, the subject is a human being.

In some embodiments of the composition of invention, optionally in combination with any of the various embodiments of invention composition herein, the bone condition is one of osteoporosis, inflammatory bone diseases, periodontal diseases, and chronic diseases-associated bone loss. An example of the inflammatory bone disease is arthritis.

In a third aspect of the present invention, it is provided a method of fabricating a composition for treating, ameliorating, or preventing a bone condition, comprising providing a biologically active agent in an effective amount for promoting bone formation and inhibiting bone resorption independent of Wnt/b-catenin signaling to promote bone repair or regeneration in a subject.

In some embodiments of the method of invention, the biologically active agent is a Wnt4 protein.

In some embodiments of the method of invention, optionally in combination with any of the various embodiments of invention method herein, the Wnt4 protein is included in a pharmaceutical composition.

In some embodiments of the method of invention, optionally in combination with any of the various embodiments of invention method herein, the pharmaceutical composition is in a formulation for systemic delivery.

In some embodiments of the method of invention, optionally in combination with any of the various embodiments of invention method herein, the pharmaceutical composition is in a formulation for local delivery.

In some embodiments of the method of invention, optionally in combination with any of the various embodiments of invention method herein, the subject is a human being.

In some embodiments of the method of invention, optionally in combination with any of the various embodiments of invention method herein, the bone condition is one of osteoporosis, inflammatory bone diseases, periodontal diseases, and chronic diseases-associated bone loss. An example of the inflammatory bone disease is arthritis.

### BRIEF DESCRIPTION OF THE DRAWINGS

FIGS. 1a-1j show Wnt4 promotes postnatal bone formation in vivo. (a) Western blot showing Wnt4 expression in primary calvarial cells extracted from WT and Wnt4 mice following osteogenic induction. HA, hemagglutinin. (b) RT-PCR analysis of Wnt4 mRNA expression in various tissues and organs. (c-e) μCT reconstruction (c), BMD (d) and BV/TV (e) of metaphyseal regions of distal femurs from 1-, 2- and 3-month-old WT and Wnt4 mice. Scale bars, 200 μm; n=12 per group. (f) H&E staining of femur sections from 1-, 2- and 3-month-old WT (n=8 per group) and Wnt4 (n=10 per group) mice. Scale bars, 300 μm. (g) Histomorphometric analysis of osteoblast counts in 3-month-old Wnt4 (n=10) versus WT (n=8) mice. Ob.S, osteoblast surface; Ob.N, osteoblast number; BS, bone surface. (h) BFR and MAR measurements from dual-fluorescent calcein labeling of 3-month-old Wnt4 (n=10) versus WT (n=8) mice. (i) Alkaline phosphatase staining of femur bone marrow MSCs from Wnt4 versus WT mice after osteogenic induction. (j) Alizarin red staining of MSCs from Wnt4 versus WT mice after osteogenic induction. Data are mean±s.d. *P<0.05, unpaired two-tailed Student's t-test.

FIGS. 2a-2j show Wnt4 attenuates osteoporosis induced by OVX. (a,b) μCT reconstruction (a) of metaphyses of distal femurs, as well as BMD and BV/TV (b), in WT versus Wnt4 mice at 2 months after OVX. Scale bars, 200 μm. (c) BFR measurement of calcein dual labeling in WT versus Wnt4 mice 2 months after OVX or sham operation. (d,e) Morphometric analysis of osteoblast (d) and osteoclast (e) counts in WT versus Wnt4 mice after OVX or sham operation. (f) TRAP staining of femur sections from WT and Wnt4 mice after OVX or sham operation. Scale bars, 30 μm. (g-i) ELISA of serum concentrations of osteocalcin (Ocn) (g), Trap5b (h) and Il-6 and Tnf (i) in WT versus Wnt4 mice after OVX or sham operation. (j) Immunostaining and quantification of active p65 in trabecular bone cells and surrounding bone marrow cells in WT and Wnt4 mice after OVX or sham operation. Scale bars, 30 μm. IOD, integral optical density. For b-e and g-j, n=8 for sham groups and n=12 for OVX groups. Data are mean±s.d. *P<0.05, **P<0.01, one-way ANOVA with Tukey's post hoc test.

FIGS. 3a-3j show Wnt4 inhibits TNF-induced bone loss and NF-κB activation. (a,b) μCT reconstruction (a) and BMD and BV/TV (b) of distal femoral metaphyseal regions from WT, Wnt4, TNFtg (TNF) and TNFtg/Wnt4 (TNF/Wnt4) mice Scale bars, 200 μm. (c) Comparisons of MAR and BFR in TNFtg mice and TNFtg/Wnt4 mice. (d,e) Morphometric analysis of osteoblast (d) and osteoclast (e) counts in TNFtg mice and TNFtg/Wnt4 mice. (f) TRAP staining of osteoclasts surrounding trabecular bones in WT, Wnt4, TNFtg and TNFtg/Wnt4 mice. Scale bars, 40 μm. (g-i) ELISA of Ocn (g), Trap5b (h) and Il-6 (i) concentrations in serum collected from WT, Wnt4, TNFtg and TNFtg/Wnt4 mice. (j) Immunostaining with antibody to active p65 and quantification of NF-κB activity surrounding the trabecular bone in WT, Wnt4, TNFtg and TNFtg/Wnt4 mice. Scale bars, 40 μm. For b-e and g-j, n=6 per group for WT and WNT4 mice and n=8 per group for TNFtg and TNFtg/Wnt mice. Data are mean±s.d. *P<0.05, **P<0.01, one-way ANOVA with Tukey's post hoc test.

FIGS. 4a-4j show Wnt4 inhibits NF-κB by interfering with Tak1-Traf6 binding. (a) Immunoblots showing the phosphorylation of Tak1, p65 and IκBα in bone marrow macrophages after treatment with Rankl, rWnt4 and rWnt4 with Rankl. (b) Immunoblots showing p65 and Tata-binding protein (Tbp) in nuclear extracts of bone marrow macrophages treated with Rankl, rWnt4 and rWnt4 with Rankl. (c) Relative NF-κB-dependent luciferase reporter activities in bone marrow macrophages after treatment with Rankl, rWnt4 and rWnt4 with Rankl. (d) Immunoblots showing the Traf6-Tak1-Tab2 complex formation induced by Rankl in bone marrow macrophages. (e) Immunoblots showing the induction of Nfatc1 expression in bone marrow macrophages after treatment with Rankl and rWnt4 with Rankl. (f) ChIP assays of the recruitment of p65 to the Nfatc1 promoter induced by Rankl Anti-IgG and primers targeting sequences 9 kb downstream of transcription start site were used as negative control. (g) ChIP assays of Nfatc1 binding to the Nfatc1 promoter. (h) Immunoblots of β-catenin in cytosolic extract (CE) and nuclear extract (NE) of bone marrow macrophages treated with Wnt3a and Wnt4. (i) Relative TOPflash luciferase activities in bone marrow macrophages treated with Wnt3a or Wnt4. (j) Real-time RT-PCR of Axin2 and Dkk1 in bone marrow macrophages treated with Wnt3a or Wnt4. For all panels with error bars, n=3 sets of cells; *P<0.05; **P<0.01, unpaired two-tailed Student's t-test. Data are mean±s.d.

FIGS. 5a-5j show rWnt4 protein attenuate established bone loss by inhibiting NF-κB. (a-c) μCT reconstruction (a), BMD and BV/TV (b) and H&E staining (c) of distal femoral metaphyseal regions from mice after sham operation, OVX and OVX with rWnt4 injection. Scale bars, 200 μm (a) and 300 μm (c). (d,e) Morphometric analysis of osteoblast (d) and osteoclast (e) counts in distal femoral metaphyses from mice after sham operation, OVX and OVX with rWnt4 injection. (f) TRAP staining showing osteoclasts surrounding trabecular bones in mice after sham operation, OVX and OVX with rWnt4 injection. Scale bars, 30 μm. (g,h) ELISA of Trap5b (g) and Ocn (h) concentrations in serum from mice after sham operation, OVX and OVX with rWnt4 injection. (i) Immunostaining with antibody to active p65 and quantification of NF-κB activity surrounding the trabecular bones from mice after sham operation, OVX and OVX with rWnt4 injection. Scale bars, 30 μm. (j) ELISA of Il-6 and Tnf concentrations in serum from mice after sham operation, OVX+PBS and OVX+rWnt4 injection. For all panels with error bars, n=8 mice for sham group; n=12 mice per group for mice receiving OVX with PBS or with rWnt4 injection. Data are mean±s.d. *P<0.05, **P<0.01, one-way ANOVA with Tukey's post hoc test.

FIGS. 6a-6g show Wnt4 promotes postnatal bone formation in vivo. (a) Southern blot of Wnt4 transgene expression in 10 founder mouse lines. (b-c) μCT analysis of BMD (b) and BV/TV (c) of 1-, 2- and 3-month-old WT and Wnt4 mice (TG-1). n=10 mice per group. *P<0.05. (d-g) Real time RT-PCR analysis of osteogenic marker genes including Runx2 (d), Sp7 (e), Ibsp (f) and Bglap (g) mRNA expression in primary bone marrow MSCs isolated from femurs of 3-month-old WT and Wnt4 mice, after osteogenic induction treatment for indicated times.

FIGS. 7a-7c show Wnt4 attenuates the expression of NF-κB-regulated molecules in vivo induced by OVX. (a-c) Immunostaining of NF-κB-dependent Tnf (a), Cox-2 (b), and Mmp9 (c) surrounding trabecular bones in the distal metaphysis of WT and Wnt4 mice two months after OVX or sham operation. Scale bars, 60 μm.

FIGS. 8a-8g show Wnt4 alleviates arthritis induced by TNF. (a-c) Photographs of hindpaws and ankle joints (a) showing swelling (yellow arrow) as well as μCT reconstruction of ankle and tibiotalar joints (c) showing bony erosions (red arrow) from 12-month-old WT, TNFtg and TNFtg/Wnt4 mice. Average arthritis scores (b) were given based on the degree of swelling and joint deviation. n=8 hindpaws for WT and TNFtg/Wnt4 groups; n=4 hindpaws for TNFtg group. **P<0.01. (d-e) H&E staining of tibiotalar (d) and interdigital (e) joints showing joint cartilage destruction and bone erosions due to invasion of inflammatory cells (black arrows). (f,g) Immunostaining of NF-κB-dependent Cox-2 (f) and Mmp9 (g) in distal femoral metaphysis of 12-month-old WT, Wnt4, TNFtg and TNFtg/Wnt4 mice. Scale bar, 1 mm (c); 200 μm (d-e); 40 μm (f-g).

FIGS. 9a-9h show Wnt4 directly inhibits osteoclast differentiation induced by Rankl (a-b) TRAP staining showing osteoclast formation from bone marrow macrophages (a) and RAW264.7 cells (b) induced by Rankl or Rankl with Wnt4. (c,d) Real time RT-PCR of Trap, Mmp9 and Ctsk mRNA in bone marrow macrophages (c) and RAW264.7 cells (d). (e,f) Real time RT-PCR of Il6 and Birc3 in bone marrow macrophages and RAW264.7 cells (f). (g) Real time RT-PCR of Tnf and Cox-2 in bone marrow macrophages. (h) Immunoblots showing the phosphorylation of p38, Jnk, and Erk of lysates from bone marrow macrophages stimulated with Rankl, Wnt4 or Rankl with Wnt4. Scale bars, 100 μm (a-b). *P<0.05; **P<0.01.

FIGS. 10a-10j show rWnt4 prevents osteoporotic bone loss by inhibiting NF-κB. (a-c) μCT reconstruction (a), BMD (b) and BV/TV (c) of distal femoral metaphysis regions from mice after sham operation, OVX and OVX immediately followed by rWnt4 injection. (d) BFR measurement from dual calcein labeling of mice. (e-f) H&E staining (e) and TRAP staining (f) in distal metaphysis of mice. (g) Morphometric analysis of osteoclast counts in distal femoral metaphysis. (h) ELISA of Trap5b concentrations in serum. (i) Immunostaining showing active p65, Tnf, Cox-2 and Mmp9 in distal femoral metaphysis. (j) ELISA of serum concentrations of Tnf and Il-6. For b, c, d, and g-j, n=8 mice for sham group; n=12 mice per group for mice receiving OVX and OVX with preventive rWnt4 injection. *P<0.05, **P<0.01, one-way ANOVA with Tukey's post hoc test. Scale bars, 200 μm (a); 300 μm (e); 25 μm (f) and (i).

FIG. 11 shows rWnt4 proteins attenuate activation of NF-κB-dependent molecules induced by OVX. Immunostaining of NF-κB-dependent Tnf, Cox-2 and Mmp9 in distal metaphysis of mice. Scale bars, 40 μm.

FIG. 12a-12k summarizes results of studies on reversal of bone loss by rWnt4 protein.

### DETAILED DESCRIPTION

### Definitions

As used herein, the term “variant” as used herein refers to a Wnt4 protein or nucleic acid that is “substantially similar” to a wild-type Wnt4 protein or Wnt4 gene. A molecule is said to be “substantially similar” to another molecule if both molecules have substantially similar structures (i.e., they are at least 50% similar in amino acid sequence as determined by BLASTp alignment set at default parameters) and are substantially similar in at least one relevant function (e.g., effect on cell migration). A variant differs from the naturally occurring Wnt4 protein or nucleic acid by one or more amino acid or nucleic acid deletions, additions, substitutions or side-chain modifications, yet retains one or more specific functions or biological activities of the naturally occurring molecule Amino acid substitutions include alterations in which an amino acid is replaced with a different naturally-occurring or a non-conventional amino acid residue. Some substitutions can be classified as “conservative,” in which case an amino acid residue contained in a Wnt4 protein is replaced with another naturally occurring amino acid of similar character either in relation to polarity, side chain functionality or size. Substitutions encompassed by variants as described herein can also be “non-conservative,” in which an amino acid residue which is present in a Wnt4 protein is substituted with an amino acid having different properties (e.g., substituting a charged or hydrophobic amino acid with an uncharged or hydrophilic amino acid), or alternatively, in which a naturally-occurring amino acid is substituted with a non-conventional amino acid. Also encompassed within the term “variant,” when used with reference to a polynucleotide or Wnt4 gene, are variations in primary, secondary, or tertiary structure, as compared to a reference polynucleotide or Wnt4 gene, respectively (e.g., as compared to a wild-type polynucleotide or Wnt4 gene). Polynucleotide changes can result in amino acid substitutions, additions, deletions, fusions and truncations in the Wnt4 protein encoded by the reference sequence.

Further, the term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

Variants can also include insertions, deletions or substitutions of amino acids, including insertions and substitutions of amino acids and other molecules) that do not normally occur in the sequence that is the basis of the variant, including but not limited to insertion of ornithine which does not normally occur in human proteins. In these embodiments, the term variant can be used interchangeably with the term “mutant” or mutation.

The term “derivative” as used herein refers to Wnt4 proteins which have been chemically modified, for example by ubiquitination, labeling, pegylation (derivatization with polyethylene glycol) or addition of other molecules. A molecule is also a “derivative” of another molecule when it contains additional chemical moieties not normally a part of the molecule. Such moieties can improve the molecule's solubility, absorption, biological half-life, etc. The moieties can alternatively decrease the toxicity of the molecule, or eliminate or attenuate an undesirable side effect of the molecule, etc. Moieties capable of mediating such effects are disclosed in Remington's Pharmaceutical Sciences, 18th edition, A. R. Gennaro, Ed., MackPubl., Easton, Pa. (1990). As such, a “derivative” polypeptide or peptide is one that is modified, for example, by glycosylation, pegylation, phosphorylation, sulfation, reduction alkylation, acylation, chemical coupling, or mild formalin treatment. A derivative may also be modified to contain a detectable label, either directly or indirectly, including, but not limited to, a radioisotope, fluorescent, and enzyme label.

The term “functional” when used in conjunction with “derivative” or “variant” refers to Wnt4 proteins which possess a biological activity that is substantially similar to a biological activity of the entity or molecule of which it is a derivative or variant. By “substantially similar” in this context is meant that at least 50% of the relevant or desired biological activity of a corresponding wild-type Wnt4 protein is retained, e.g., preferably the variant retains at least 60%, at least 70%, at least 80%, at least 90%, at least 95%, at least 100% or even higher (i.e., the variant or derivative has greater activity than the wild-type), e.g., at least 110%, at least 120%, or more compared to a measurable activity of the wild-type Wnt4 protein.

The term “therapeutically effective amount”, as used herein, is an amount of an agent that is sufficient to produce a statistically significant, measurable change of a condition in repaired tissue using the agent disclosed herein as compared with the condition in the repaired tissue without using the agent. Such effective amounts can be gauged in clinical trials as well as animal studies. Such a statistically significant, measurable, and positive change of a condition in repaired tissue using the agent disclosed herein as compared with the condition in the repaired tissue without using the agent is referred to as being an “improved condition”.

As used herein, the term “significantly” or “significant” shall mean statistically significant.

As used herein, the term “agent” refers to a biologically active agent in an effective amount for promoting bone formation and inhibiting bone resorption in the animal independent of Wnt/b-catenin signaling. An example of the agent is a Wnt4 proteins or a variant or derivative or analog thereof. In some embodiments, the term also encompasses a PEGylated Wnt4 protein or a Wnt4 protein bearing a short alkyl chain, a short polymer chain, a short poly(amino acid) chain, or acyl group such as methyl or ethyl or acetyl, for example.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of refers to those elements required for a given embodiment. The term permits the presence of elements that do not materially affect the basic and novel or functional characteristic(s) of that embodiment of the invention.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “induces or enhances an immune response” is meant causing a statistically significant induction or increase in an immune response over a control sample to which the peptide, polypeptide or protein has not been administered. Preferably the induction or enhancement of the immune response results in a prophylactic or therapeutic response in a subject. Examples of immune responses are increased production of type I IFN, increased resistance to viral and other types of infection by alternate pathogens. The enhancement of immune responses to tumors (anti-tumor responses), or the development of vaccines to prevent tumors or eliminate existing tumors.

The term “active fragment or variant” is meant a fragment that is 100% identical to a contiguous portion of the peptide, polypeptide or protein, or a variant that is at least 90%, preferably 95% identical to a fragment up to and including the full length peptide, polypeptide or protein. A variant, for example, may include conservative amino acid substitutions, as defined in the art, or nonconservative substitutions, providing that at least e.g. 10%, 25%, 50%, 75% or 90% of the activity of the original peptide, polypeptide or protein is retained. Also included are Wnt4 protein mutant molecules, fragments or variants having post-translational modifications such as sumoylation, phosphorylation glycosylation, splice variants, and the like, all of which may effect the efficacy of Wnt4 protein of invention function and/or activity, both known and yet to be discovered.

Unless otherwise indicated, the terms “peptide”, “polypeptide” or “protein” are used interchangeably herein, although typically they refer to peptide sequences of varying sizes.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) or single base mutations in which the polynucleotide sequence varies by one base. The presence of SNPs may be indicative of, for example, a certain population with a propensity for a disease state, that is susceptibility versus resistance.

Derivative polynucleotides include nucleic acids subjected to chemical modification, for example, replacement of hydrogen by an alkyl, acyl, or amino group. Derivatives, e.g., derivative oligonucleotides, may comprise non-naturally-occurring portions, such as altered sugar moieties or inter-sugar linkages. Exemplary among these are phosphorothioate and other sulfur containing species which are known in the art. Derivative nucleic acids may also contain labels, including radionucleotides, enzymes, fluorescent agents, chemiluminescent agents, chromogenic agents, substrates, cofactors, inhibitors, magnetic particles, and the like.

The term “immunoregulatory” is meant a compound, composition or substance that is immunogenic (i.e. stimulates or increases an immune response) or immunosuppressive (i.e. reduces or suppresses an immune response).

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

By “encoding” or “encoded”, “encodes”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code.

Whenever referred to herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

The terms “patient”, “subject” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests. For example the term “treat” or “treating” with respect to tumor cells refers to stopping the progression of said cells, slowing down growth, inducing regression, or amelioration of symptoms associated with the presence of said cells. Treatment of an individual suffering from an infectious disease organism refers to a decrease and elimination of the disease organism from an individual. For example, a decrease of viral particles as measured by plaque forming units or other automated diagnostic methods such as ELISA etc.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

### Wnt/Beta-Catenin Signaling

The wnt signal transduction cascade controls myriad biological phenomena throughout development and adult life of all animals. In parallel, aberrant Wnt signaling underlies a wide range of pathologies in humans (Cell, Volume 149, Issue 6, 1192-1205, 8 Jun. 2012).

The conserved Wnt/β-Catenin pathway regulates stem cell pluripotency and cell fate decisions during development. This developmental cascade integrates signals from other pathways, including retinoic acid, FGF, TGF-β, and BMP, within different cell types and tissues. The Wnt ligand is a secreted glycoprotein that binds to Frizzled receptors, which triggers displacement of the multifunctional kinase GSK-3β from a regulatory APC/Axin/GSK-3β-complex. In the absence of Wnt-signal (Off-state), β-catenin, an integral E-cadherin cell-cell adhesion adaptor protein and transcriptional co-regulator, is targeted by coordinated phosphorylation by CK1 and the APC/Axin/GSK-3β-complex leading to its ubiquitination and proteasomal degradation through the β-TrCP/SKP pathway. In the presence of Wnt ligand (On-state), the co-receptor LRP5/6 is brought in complex with Wnt-bound Frizzled. This leads to activation of Dishevelled (Dvl) by sequential phosphorylation, poly-ubiquitination, and polymerization, which displaces GSK-3β from APC/Axin through an unclear mechanism that may involve substrate trapping and/or endosome sequestration. The transcriptional effects of Wnt ligand is mediated via Rac1-dependent nuclear translocation of β-catenin and the subsequent recruitment of LEF/TCF DNA-binding factors as co-activators for transcription, acting partly by displacing Groucho-HDAC co-repressors. Additionally, β-catenin has also been shown to cooperate with the homeodomain factor Prop1 in context-dependent activation as well as repression complexes. Importantly, researchers have found β-catenin point mutations in human tumors that prevent GSK-3β phosphorylation and thus lead to its aberrant accumulation. E-cadherin, APC, and Axin mutations have also been documented in tumor samples, underscoring the deregulation of this pathway in cancer. Furthermore, GSK-3β is involved in glycogen metabolism and other signaling pathways, which has made its inhibition relevant to diabetes and neurodegenerative disorders. See, e.g., Angers S, Moon R T (2009) Proximal events in Wnt signal transduction. Nat. Rev. Mol. Cell Biol. 10(7), 468-77; Clevers H, Nusse R (2012) Wnt/β-catenin signaling and disease. Cell 149(6), 1192-205.

### Bone Formation and Osteoblast

Bone formation is a dynamic process where osteoblasts are responsible for bone formation and osteoclasts for its resorptio (Caetino-Lopez, J., et al., Acta Reumatol Port. 2007 April-June; 32(2):103-10). Osteoblasts are specialized mesenchymal cells that undergo a process of maturation where genes like core-binding factor alpha1 (Cbfa1) and osterix (Osx) play a very important role. Moreover, it was found recently that Wnt/beta-catenin pathway plays a part on osteoblast differentiation and proliferation. In fact, mutations on some of the proteins involved in this pathway, like the low-density lipoprotein receptor related protein 5/6 (LRP5/6) lead to bone diseases. Osteoblast have also a role in regulation of bone resorption through receptor activator of nuclear factor-kappaB (RANK) ligand (RANKL), that links to its receptor, RANK, on the surface of pre-osteoblast cells, inducing their differentiation and fusion. On the other hand, osteoblasts secrete a soluble decoy receptor (osteoprotegerin, OPG) that blocks RANK/RANKL interaction by binding to RANKL and, thus, prevents osteoclast differentiation and activation. Therefore, the balance between RANKL and OPG determines the formation and activity of osteoclasts. Another factor that influences bone mass is leptin, a hormone produced by adipocytes that have a dual effect. It can act through the central nervous system and diminish osteoblasts activity, or can have an osteogenic effect by binding directly to its receptors on the surface of osteoblast cells.

### Bone Resorption and Osteoclast

Bone resorption is the process by which osteoclasts break down bone and release the minerals, resulting in a transfer of calcium from bone fluid to the blood (see, e.g., Teitelbaum S L. (2000). “Bone resorption by osteoclasts.”. Science 289: 1504-8).

The osteoclasts are multi-nucleated cells that contain numerous mitochondria and lysosomes. These are the cells responsible for the resorption of bone. Osteoclasts are generally present on the outer layer of bone, just beneath the periosteum. Attachment of the osteoclast to the osteon begins the process. The osteoclast then induces an infolding of its cell membrane and secretes collagenase and other enzymes important in the resorption process. High levels of calcium, magnesium, phosphate and products of collagen will be released into the extracellular fluid as the osteoclasts tunnel into the mineralized bone. Osteoclasts are also prominent in the tissue destruction commonly found in psoriatic arthritis and other rheumatology related disorders.

Bone resorption can also be the result of disuse and the lack of stimulus for bone maintenance. Astronauts, for instance will undergo a certain amount of bone resorption due to the lack of gravity providing the proper stimulus for bone maintenance.

During childhood, bone formation exceeds resorption, but as the aging process occurs, resorption exceeds formation.

Bone resorption is highly constructable stimulated or inhibited by signals from other parts of the body, depending on the demand for calcium. Calcium-sensing membrane receptors in the parathyroid gland monitor calcium levels in the extracellular fluid. Low levels of calcium stimulates the release of parathyroid hormone (PTH) from chief cells of the parathyroid gland. In addition to its effects on kidney and intestine, PTH also increases the number and activity of osteoclasts to draw calcium from bone, and thus stimulates bone resorption.

High levels of calcium in the blood, on the other hand, leads to decreased PTH release from the parathyroid gland, decreasing the number and activity of osteoclasts, resulting in less bone resorption.

In some cases where bone resorption becomes accelerated, the bone is broken down much faster than it can be renewed. The bone becomes more porous and fragile, exposing people to the risk of fractures. Depending on where in the body bone resorption occurs, additional problems like tooth loss can also arise. Some people who experience bone resorption are astronauts. Due to the condition of being in a zero-gravity environment, astronauts do not need to work their musculoskeletal system as hard as those in a typical environment. The body responds with bone resorption, causing a loss in bone density.

### Compositions

In one aspect of the present invention, it is provided a composition for bone regeneration in a subject, comprising a biologically active agent in an effective amount for promoting bone formation and inhibiting bone resorption in the animal independent of Wnt/b-catenin signaling.

In some embodiments of the composition of invention, the biologically active agent is a Wnt4 protein.

In some embodiments of the composition of invention, optionally in combination with any of the various embodiments of invention composition herein, the Wnt4 protein is included in a pharmaceutical composition.

In some embodiments of the composition of invention, optionally in combination with any of the various embodiments of invention composition herein, the pharmaceutical composition is in a formulation for systemic delivery.

In some embodiments of the method of composition, optionally in combination with any of the various embodiments of invention composition herein, the composition comprises a gene construct encoding the Wnt4 protein.

In some embodiments of the method of composition, optionally in combination with any of the various embodiments of invention composition herein, the composition comprises an mRNA encoding the Wnt4 protein.

In some embodiments of the composition of invention, optionally in combination with any of the various embodiments of invention composition herein, the pharmaceutical composition is in a formulation for local delivery.

In some embodiments of the method of invention, optionally in combination with any of the various embodiments of invention composition herein, the subject is a human being.

In some embodiments of the composition of invention, optionally in combination with any of the various embodiments of invention composition herein, the bone condition is one of osteoporosis, inflammatory bone diseases, periodontal diseases, and chronic diseases-associated bone loss. An example of the inflammatory bone disease is arthritis.

### Other Agents

In some embodiments, the pharmaceutical composition described herein may include a Wnt4 protein and other agents effective for promoting bone generation. Such other agents include, e.g., a bone morphogenetic protein (BMP) such as BMP-1, BMP-2, BMP-3, BMP-4, BMP-5, BMP-6, BMP-7, BMP-8, BMP-9, BMP-10, BMP-11, BMP-12, BMP-13, BMP-14, BMP-15, BMP-16, BMP-17, BMP-18, BMP-19, BMP-20, BMP-21, FGF (fibroblast growth factors, e.g., FGF1 FGF2, FGF4, FGF7, FGF10, FGF19, FGF21, FGF23), TGF-.beta. (transforming growth factor-.beta., e.g., TGF-.beta.1), IGF (insulin-like growth factor, e.g., IGF-I), VEGF (vascular endothelial growth factor), PDGF (platelet-derived growth factor), PTH (parathyroid hormone)/PTHrp (PTH-regulated protein), oxysterols, lipophilic statins, growth/differentiation factor 5 (GDF5); and LIM mineralization proteins (LMPS) of which at least three splice variants exist. Some studies concerning these factors and mechanisms through which they act are described in Nakashima, K. and B. de Crombrugghe, Trends Genet, 2003. 19(S): p. 458-66; Tou, L., N. Quibria, and J. M. Alexander, Mol Cell Endocrinol, 2003. 205(1-2): p. 121-9; Pei, Y., et al., Acta Pharmacol Sin, 2003. 24(10): p. 975-84; Lee, M. H., et al., J Cell Biochem, 1999. 73(1): p. 114-25; Franceschi, R. T. and G. Xiao, J Cell Biochem, 2003. 88(3): p. 446-54; Kim, H. J., et al., J Biol Chem, 2003. 278(1): p. 319-26; Zelzer, E., et al., Mech Dev, 2001. 106(1-2): p. 97-106; Himeno, M., et al., J Bone Miner Res, 2002. 17(7): p. 1297-305; Kha, H. T. et al. J Bone Miner Res 19, 830-40, 2004; Izumo, N. et al. Methods Find Exp Clin Pharmacol 23, 389-94, 2001; Hatakeyama, Y. et al. J Cell Biochem 91, 1204-17, 2004; Pola, E. et al. Gene Ther 11, 683-93, 2004). One study reported that activating mutations in FGF receptor1 (FGFR1) dramatically increased Cbfa1 expression, osteoblast proliferation and differentiation, and bony calvarial overgrowth across cranial sutures in mice (Zhou, Y. X., et al., Hun Mol Genet, 2000. 9(13): p. 2001-8).

In some embodiments, the composition described herein can specifically exclude one or more the above described agents.

### Formulation Carriers

The pharmaceutical composition described herein may be administered to a subject in need of treatment by a variety of routes of administration, including orally and parenterally, (e.g., intravenously, subcutaneously or intramedullary), intranasally, as a suppository or using a “flash” formulation, i.e., allowing the medication to dissolve in the mouth without the need to use water, topically, intradermally, subcutaneously and/or administration via mucosal routes in liquid or solid form. The pharmaceutical composition can be formulated into a variety of dosage forms, e.g., extract, pills, tablets, microparticles, capsules, oral liquid.

There may also be included as part of the pharmaceutical composition pharmaceutically compatible binding agents, and/or adjuvant materials. The active materials can also be mixed with other active materials including antibiotics, antifungals, other virucidals and immunostimulants which do not impair the desired action and/or supplement the desired action.

In one embodiment, the mode of administration of the pharmaceutical composition described herein is oral. Oral compositions generally include an inert diluent or an edible carrier. They may be enclosed in gelatin capsules or compressed into tablets. For the purpose of oral therapeutic administration, the aforesaid compounds may be incorporated with excipients and used in the form of tablets, troches, capsules, elixirs, suspensions, syrups, wafers, chewing gums and the like. Some variation in dosage will necessarily occur, however, depending on the condition of the subject being treated. These preparations should produce a serum concentration of active ingredient of from about 0.01 nM to 1,000,000 nM, e.g., from about 0.2 to 40 .mu.M. A preferred concentration range is from 0.2 to 20 .mu.M and most preferably about 1 to 10 .mu.M. However, the concentration of active ingredient in the drug composition itself depends on bioavailability of the drug and other factors known to those of skill in the art.

In another embodiment, the mode of administration of the pharmaceutical compositions described herein is topical or mucosal administration. A specifically preferred mode of mucosal administration is administration via female genital tract. Another preferred mode of mucosal administration is rectal administration.

Various polymeric and/or non-polymeric materials can be used as adjuvants for enhancing mucoadhesiveness of the pharmaceutical composition disclosed herein. The polymeric material suitable as adjuvants can be natural or synthetic polymers. Representative natural polymers include, for example, starch, chitosan, collagen, sugar, gelatin, pectin, alginate, karya gum, methylcellulose, carboxymethylcellulose, methylethylcellulose, and hydroxypropylcellulose. Representative synthetic polymers include, for example, poly(acrylic acid), tragacanth, poly(methyl vinylether-co-maleic anhydride), poly(ethylene oxide), carbopol, poly(vinyl pyrrolidine), poly(ethylene glycol), poly(vinyl alcohol), poly(hydroxyethylmethylacrylate), and polycarbophil. Other bioadhesive materials available in the art of drug formulation can also be used (see, for example, Bioadhesion—Possibilities and Future Trends, Gurny and Junginger, eds., 1990).

It is to be noted that dosage values also varies with the specific severity of the disease condition to be alleviated. It is to be further understood that for any particular subject, specific dosage regimens should be adjusted to the individual need and the professional judgment of the person administering or supervising the administration of the aforesaid compositions. It is to be further understood that the concentration ranges set forth herein are exemplary only and they do not limit the scope or practice of the invention. The active ingredient may be administered at once, or may be divided into a number of smaller doses to be administered at varying intervals of time.

The formulation may contain the following ingredients: a binder such as microcrystalline cellulose, gum tragacanth or gelatin; an excipient such as starch or lactose, a disintegrating agent such as alginic acid, Primogel, corn starch and the like; a lubricant such as magnesium stearate or Sterotes; a glidant such as colloidal silicon dioxide; and a sweetening agent such as sucrose or saccharin or flavoring agent such as peppermint, methyl salicylate, or orange flavoring may be added. When the dosage unit form is a capsule, it may contain, in addition to material of the above type, a liquid carrier such as a fatty oil. Other dosage unit forms may contain other various materials which modify the physical form of the dosage unit, for example, as coatings. Thus tablets or pills may be coated with sugar, shellac, or other enteric coating agents. Materials used in preparing these various compositions should be pharmaceutically pure and non-toxic in the amounts used.

The solutions or suspensions may also include the following components: a sterile diluent such as water for injection, saline solution, fixed oils, polyethylene glycols, glycerine, propylene glycol or other synthetic solvents; antibacterial agents such as benzyl alcohol or methylparabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

The pharmaceutical compositions of the present invention are prepared as formulations with pharmaceutically acceptable carriers. Preferred are those carriers that will protect the active compound against rapid elimination from the body, such as a controlled release formulation, including implants and microencapsulated delivery systems. Biodegradable, biocompatable polymers can be used, such as polyanhydrides, polyglycolic acid, collagen, and polylactic acid. Methods for preparation of such formulations can be readily performed by one skilled in the art.

Liposomal suspensions (including liposomes targeted to infected cells with monoclonal antibodies to viral antigens) are also preferred as pharmaceutically acceptable carriers. Methods for encapsulation or incorporation of compounds into liposomes are described by Cozzani, I.; Joni, G.; Bertoloni, G.; Milanesi, C.; Sicuro, T. Chem. Biol. Interact. 53, 131-143 (1985) and by Jori, G.; Tomio, L.; Reddi, E.; Rossi, E. Br. J. Cancer 48, 307-309 (1983). These may also be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

Other methods for encapsulating compounds within liposomes and targeting areas of the body are described by Sicuro, T.; Scarcelli, V.; Vigna, M. F.; Cozzani, I. Med. Biol. Environ. 15(1), 67-70 (1987) and Joni, G.; Reddi, E.; Cozzani, I.; Tomio, L. Br. J. Cancer, 53(5), 615-21 (1986).

The pharmaceutical composition described herein may be administered in single (e.g., once daily) or multiple doses or via constant infusion. The compounds of this invention may also be administered alone or in combination with pharmaceutically acceptable carriers, vehicles or diluents, in either single or multiple doses. Suitable pharmaceutical carriers, vehicles and diluents include inert solid diluents or fillers, sterile aqueous solutions and various organic solvents. The pharmaceutical compositions formed by combining the compounds of this invention and the pharmaceutically acceptable carriers, vehicles or diluents are then readily administered in a variety of dosage forms such as tablets, powders, lozenges, syrups, injectable solutions and the like. These pharmaceutical compositions can, if desired, contain additional ingredients such as flavorings, binders, excipients and the like according to a specific dosage form.

Thus, for example, for purposes of oral administration, tablets containing various excipients such as sodium citrate, calcium carbonate and/or calcium phosphate may be employed along with various disintegrants such as starch, alginic acid and/or certain complex silicates, together with binding agents such as polyvinylpyrrolidone, sucrose, gelatin and/or acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions or elixirs are desired for oral administration, the active pharmaceutical agent therein may be combined with various sweetening or flavoring agents, coloring matter or dyes and, if desired, emulsifying or suspending agents, together with diluents such as water, ethanol, propylene glycol, glycerin and/or combinations thereof.

For parenteral administration, solutions of the compounds of this invention in sesame or peanut oil, aqueous propylene glycol, or in sterile aqueous solutions may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, the sterile aqueous media employed are all readily available by standard techniques known to those skilled in the art.

For intranasal administration or administration by inhalation, the compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of a compound of this invention. Capsules and cartridges (made, for example, from gelatin) for use in an inhaler or insufflator may be formulated containing a powder mix of a compound or compounds of the invention and a suitable powder base such as lactose or starch.

The pharmaceutical composition provided herein can also be used with another pharmaceutically active agent effective for a disease such as neurodisorders, cardiovascular disorders, tumors, AIDS, depression, and/or type-1 and type-2 diabetes. Such additional agents can be, for example, antiviral agent, antibiotics, anti-depression agent, anti-cancer agents, immunosuppressant, anti-fungal, and a combination thereof.

The pharmaceutical composition described herein can be formulated alone or together with the other agent in a single dosage form or in a separate dosage form. Methods of preparing various pharmaceutical formulations with a certain amount of active ingredient are known, or will be apparent in light of this disclosure, to those skilled in this art. For examples of methods of preparing pharmaceutical formulations, see Remington's Pharmaceutical Sciences, Mack Publishing Company, Easton, Pa., 19th Edition (1995).

### Scaffolds

In one embodiment, the composition of invention can be formulated into a scaffold. Such a scaffold can include a carrier, which can be biodegradable, such as degradable by enzymatic or hydrolytic mechanisms. Examples of carriers include, but are not limited to synthetic absorbable polymers such as such as but not limited to poly(.alpha.-hydroxy acids) such as poly (L-lactide) (PLLA), poly (D, L-lactide) (PDLLA), polyglycolide (PGA), poly (lactide-co-glycolide (PLGA), poly (-caprolactone), poly (trimethylene carbonate), poly (p-dioxanone), poly (-caprolactone-co-glycolide), poly (glycolide-co-trimethylene carbonate) poly (D, L-lactide-co-trimethylene carbonate), polyarylates, polyhydroxybutyrate (PHB), polyanhydrides, poly (anhydride-co-imide), propylene-co-fumarates, polylactones, polyesters, polycarbonates, polyanionic polymers, polyanhydrides, polyester-amides, poly(amino-acids), homopolypeptides, poly(phosphazenes), poly (glaxanone), polysaccharides, and poly(orthoesters), polyglactin, polyglactic acid, polyaldonic acid, polyacrylic acids, polyalkanoates; copolymers and admixtures thereof, and any derivatives and modifications. See for example, U.S. Pat. No. 4,563,489, and PCT Int. Appl. # WO/03024316, herein incorporated by reference. Other examples of carriers include cellulosic polymers such as, but not limited to alkylcellulose, hydroxyalkylcellulose, methylcellulose, ethylcellulose, hydroxyethylcellulose, hydroxypropylcellulose, hydroxypropyl-methylcellulose, carboxymethylcellulose, and their cationic salts. Other examples of carriers include synthetic and natural bioceramics such as, but not limited to calcium carbonates, calcium phosphates, apatites, bioactive glass materials, and coral-derived apatites.

In one embodiment, the carrier may further be coated by compositions, including bioglass and or apatites derived from sol-gel techniques, or from immersion techniques such as, but not limited to simulated body fluids with calcium and phosphate concentrations ranging from about 1.5 to 7-fold the natural serum concentration and adjusted by various means to solutions with pH range of about 2.8-7.8 at temperature from about 15-65 degrees C. Other examples of carriers include collagen (e.g. Collastat, Helistat collagen sponges), hyaluronan, fibrin, chitosan, alginate, and gelatin, or a mixture thereof.

In one embodiment, the carrier may include heparin-binding agents; including but not limited to heparin-like polymers e.g. dextran sulfate, chondroitin sulfate, heparin sulfate, fucan, alginate, or their derivatives; and peptide fragments with amino acid modifications to increase heparin affinity. See for example, Journal of Biological Chemistry (2003), 278(44), p. 43229-43235, the teachings of which are incorporated herein by reference.

In one embodiment, the scaffold may be in the form of a liquid, solid or gel.

In one embodiment, the scaffold can be a carrier that is in the form of a flowable gel. The gel may be selected so as to be injectable, such as via a syringe at the site where bone formation is desired. The gel may be a chemical gel which may be a chemical gel formed by primary bonds, and controlled by pH, ionic groups, and/or solvent concentration. The gel may also be a physical gel which may be formed by secondary bonds and controlled by temperature and viscosity. Examples of gels include, but are not limited to, pluronics, gelatin, hyaluronan, collagen, polylactide-polyethylene glycol solutions and conjugates, chitosan, chitosan & b-glycerophosphate (BST-gel), alginates, agarose, hydroxypropyl cellulose, methyl cellulose, polyethylene oxide, polylactides/glycolides in N-methyl-2-pyrrolidone. See for example, Anatomical Record (2001), 263(4), 342-349, the teachings of which are incorporated herein by reference.

In one embodiment of the scaffold, the carrier may be photopolymerizable, such as by electromagnetic radiation with wavelength of at least about 250 nm. Example of photopolymerizable polymers include polyethylene (PEG) acrylate derivatives, PEG methacrylate derivatives, propylene fumarate-co-ethylene glycol, polyvinyl alcohol derivatives, PEG-co-poly(-hydroxy acid) diacrylate macromers, and modified polysaccharides such as hyaluronic acid derivatives and dextran methacrylate.

In one embodiment, the scaffold may include a carrier that is temperature sensitive. Examples include carriers made from N-isopropylacrylamide (NiPAM), or modified NiPAM with lowered lower critical solution temperature (LCST) and enhanced peptide (e.g. NELL1) binding by incorporation of ethyl methacrylate and N-acryloxysuccinimide; or alkyl methacrylates such as butylmethacrylate, hexylmethacrylate and dodecylmethacrylate (PCT Int. Appl. WO/2001070288; U.S. Pat. No. 5,124,151, the teachings of which are incorporated herein by reference).

In one embodiment of the scaffold, where the carrier may have a surface that is decorated and/or immobilized with cell adhesion molecules, adhesion peptides, and adhesion peptide analogs which may promote cell-matrix attachment via receptor mediated mechanisms, and/or molecular moieties which may promote adhesion via non-receptor mediated mechanisms binding such as, but not limited to polycationic polyamino-acid-peptides (e.g. poly-lysine), polyanionic polyamino-acid-peptides, Mefp-class adhesive molecules and other DOPA-rich peptides (e.g. poly-lysine-DOPA), polysaccharides, and proteoglycans. See for example, PCT Int. Appl. WO/2004005421; WO/2003008376; WO/9734016, the teachings of which are incorporated herein by reference.

In one embodiment of the scaffold, the carrier may be comprised of sequestering agents such as, but not limited to, collagen, gelatin, hyaluronic acid, alginate, poly(ethylene glycol), alkylcellulose (including hydroxyalkylcellulose), including methylcellulose, ethylcellulose, hydroxyethylcellulose, hydroxypropylcellulose, hydroxypropyl-methylcellulose, and carboxymethylcellulose, blood, fibrin, polyoxyethylene oxide, calcium sulfate hemihydrate, apatites, carboxyvinyl polymer, and poly(vinyl alcohol). See for example, U.S. Pat. No. 6,620,406, herein incorporated by reference.

In one embodiment of the scaffold, the carrier may include buffering agents such as, but not limited to glycine, glutamic acid hydrochloride, sodium chloride, guanidine, heparin, glutamic acid hydrochloride, acetic acid, succinic acid, polysorbate, dextran sulfate, sucrose, and amino acids. See for example, U.S. Pat. No. 5,385,887, herein incorporated by reference. In one embodiment, the carrier may include a combination of materials such as those listed above. By way of example, the carrier may be a PLGA/collagen carrier membrane.

In one embodiment, the scaffold can be an implant of the various embodiments described herein.

### Time Release Formulation

In one embodiment, the composition according to this invention may be contained within a time release tablet. A bioactive agent described herein (e.g. a Wnt4 protein) can be formulated with an acceptable carrier to form a pharmacological composition. Acceptable carriers can contain a physiologically acceptable compound that acts, for example, to stabilize the composition or to increase or decrease the absorption of the agent. Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the anti-mitotic agents, or excipients or other stabilizers and/or buffers. Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of a carrier, including a physiologically acceptable compound depends, for example, on the route of administration.

### Dosages

The composition of invention can have a dosage of about 1 ng to about 500 mg, for example, about 10 ng, 20 ng, 50 ng, 100 ng, 200 ng, 500 ng, 1 micro gram, about 10 micro gram, about 50 micro gram, about 100 micro gram, about 200 micro gram, about 500 micro gram, or about 1 mg.

### Dosage Forms

Embodiments of the composition of invention can be administered in a variety of unit dosage forms depending upon the method of administration. For example, unit dosage forms suitable may include powder, tablets, pills, capsules.

### Method of Use

In one aspect of the present invention, it is provided a method for treating, ameliorating, or preventing a bone condition, comprising administering to a subject in need thereof a biologically active agent in an effective amount for promoting bone formation and inhibiting bone resorption independent of Wnt/b-catenin signaling to promote bone repair or regeneration in the subject.

In some embodiments of the method of invention, the biologically active agent is a Wnt4 protein.

In some embodiments of the method of invention, optionally in combination with any of the various embodiments of invention method herein, the Wnt4 protein is included in a pharmaceutical composition.

In some embodiments of the method of invention, optionally in combination with any of the various embodiments of invention method herein, administering comprising administering to the subject a gene construct encoding the Wnt4 protein.

In some embodiments of the method of invention, optionally in combination with any of the various embodiments of invention method herein, administering comprising transfecting a cell of the subject a gene construct encoding the Wnt4 protein.

In some embodiments of the method of invention, optionally in combination with any of the various embodiments of invention method herein, administering comprises administering to the subject an mRNA encoding the Wnt4 protein.

In some embodiments of the method of invention, optionally in combination with any of the various embodiments of invention method herein, the pharmaceutical composition is in a formulation for systemic delivery.

In some embodiments of the method of invention, optionally in combination with any of the various embodiments of invention method herein, the pharmaceutical composition is in a formulation for local delivery.

In some embodiments of the method of invention, optionally in combination with any of the various embodiments of invention method herein, the subject is a human being.

In some embodiments of the method of invention, optionally in combination with any of the various embodiments of invention method herein, the bone condition is one of osteoporosis, inflammatory bone diseases, periodontal diseases, and chronic diseases-associated bone loss. An example of the inflammatory bone disease is arthritis and periodontal diseases.

### Method of Making

In a third aspect of the present invention, it is provided a method of fabricating a composition for treating, ameliorating, or preventing a bone condition, comprising providing a biologically active agent in an effective amount for promoting bone formation and inhibiting bone resorption independent of Wnt/b-catenin signaling to promote bone repair or regeneration in a subject.

In some embodiments of the method of invention, the biologically active agent is a Wnt4 protein.

In some embodiments of the method of invention, optionally in combination with any of the various embodiments of invention method herein, the Wnt4 protein is included in a pharmaceutical composition.

In some embodiments of the method of invention, optionally in combination with any of the various embodiments of invention method herein, the pharmaceutical composition is in a formulation for systemic delivery.

In some embodiments of the method of invention, optionally in combination with any of the various embodiments of invention method herein, the pharmaceutical composition is in a formulation for local delivery.

In some embodiments of the method of invention, optionally in combination with any of the various embodiments of invention method herein, the subject is a human being.

In some embodiments of the method of invention, optionally in combination with any of the various embodiments of invention method herein, the bone condition is osteoporosis or arthritis.

### Wnt4 Proteins Preparation

An example of recombinant production of Wnt family proteins, including Wnt4 proteins, is described in U.S. Pat. No. 7,175,842, the teaching of which is incorporated herein by reference in its entirety. In some embodiments, Wnt4 protein can be a Wnt4 peptide, which, as used herein, includes a shorter amino acid sequence than Wnt4 protein. In some further embodiments, the Wnt4 peptide can be a Wnt4 peptide mimetics.

### Peptide Synthesis

Before the peptide synthesis starts, the amine terminus of the amino acid (starting material) can protected with FMOC (9-fluoromethyl carbamate) or other protective groups, and a solid support such as a Merrifield resin (free amines) is used as an initiator. Then, step (1) through step (3) reactions are performed and repeated until the desired peptide is obtained: (1) a free-amine is reacted with carboxyl terminus using carbodiimide chemistry, (2) the amino acid sequence is purified, and (3) the protecting group, e.g., the FMOC protecting group, is removed under mildly acidic conditions to yield a free amine. The peptide can then be cleaved from the resin to yield a free standing peptide or peptide mimetics.

### EXAMPLES

The embodiments of the present invention will be illustrated by the following set forth examples. All parameters and data are not to be construed to unduly limit the scope of the embodiments of the invention.

### Example 1

### Studies on Prevention of Bone Loss and Inflammation by Wnt4 Signaling Through Inhibiting Nuclear Factor-κB

### Introduction

Osteoporosis and inflammation-related bone loss affect millions of people worldwide. Chronic inflammation associated with aging promotes bone resorption and impairs bone formation, Here we show that Wnt4 attenuates bone loss in osteoporosis and inflammation mouse models by inhibiting nuclear factor-κB (NF-κB) via noncanonical Wnt signaling. Transgenic mice expressing Wnt4 from osteoblasts were significantly protected from bone loss and chronic inflammation induced by ovariectomy or tumor necrosis factor. In addition to promoting bone formation, Wnt4 inhibited osteoclast formation and bone resorption. Mechanistically, Wnt4 inhibited NF-κB activation mediated by transforming growth factor-β-activated kinase-1 (Tak1) in macrophages and osteoclast precursors independently of β-catenin. Moreover, recombinant Wnt4 alleviated bone loss and inflammation by inhibiting NF-κB in vivo in mouse models of bone disease. Given its dual role in promoting bone formation and inhibiting bone resorption, our results suggest that Wnt4 signaling could be an attractive therapeutic target for treating osteoporosis.

Normal bone remodeling maintains constant bone mass by an orchestrated balance between the destruction of preexisting bone by osteoclasts and rebuilding it by osteoblasts^{1,2}. Osteoporosis, the most common metabolic bone disease, is closely associated with advanced age and increased proinflammatory cytokine levels in bone marrow microenvironment³⁻⁸. It has become a leading cause of morbidity and mortality in our aging population⁹⁻¹¹. In osteoporosis, bone homeostasis is dysregulated by hormonal deficiency and aging, leading to increased bone turnover with enhanced bone formation and even greater rates of bone resorption, resulting in a net bone loss. This imbalance in bone remodeling is also a hallmark in other aging-related bone pathologies, such as reduced formation and accelerated resorption in inflammatory bone diseases and low bone turnover in physiological aging. Both bone formation and resorption are regulated on the local level by factors secreted by bone cells, as well as on the systemic level by hormones^{4,11-14.}

Chronic inflammation has been found to be associated with osteoporosis^{1,6,7}. In general, the transcription factor NF-κB is activated during inflammatory processes¹⁵. Growing evidence suggests that NF-κB plays an important role in aging-related disorders, including aging-related bone loss and osteoporosis¹⁶⁻¹⁹ Inhibition of NF-κB has been shown to attenuate osteoporosis and arthritis^{20,21}. We previously reported that NF-κB activation inhibits bone formation in estrogen deficiency-induced bone loss²². Thus, targeting NF-κB may allow both inhibition of bone resorption and promotion of bone formation.

The Wnt family proteins are key regulators in growth and development, stem cell self-renewal and cancer development^{23,24}. Wnt signaling has also emerged as a critical player in bone homeostasis^{25,26}. The 19 Wnt family proteins are divided into canonical and noncanonical ligands based on their dependence on transduction through β-catenin²⁷⁻²⁹. Although there have been a few studies elucidating the role of noncanonical Wnt signaling in osteoblast differentiation³⁰⁻³³, little is known regarding how this signaling pathway affects osteoclast formation. Signaling between Wnt5a and receptor tyrosine kinase-like orphan receptor-2 (Ror2) has been found to promote osteoclastogenesis by activating the Wnt-c-Jun terminal kinase (Jnk) pathway³². Previously, we found that Wnt4, a prototypical ligand for the noncanonical Wnt pathway, is able to promote osteoblast differentiation of mesenchymal stem cells (MSCs)³³. To further explore the therapeutic potential of Wnt4, we generated transgenic mice that express Wnt4 in osteoblasts. We found that in addition to enhancing bone formation in vivo, Wnt4 could inhibit osteoclast formation and inflammation in vivo, thus attenuating bone loss and osteoporosis. Together with our previously published findings, our results suggest that Wnt4 signaling may represent an attractive target to treat bone loss as it promotes osteoblast generation and inhibits osteoclast formation.

### Methods

### Generation of Transgenic Mice and Experimental Animals.

We used the plasmid pGL647, which contained the Col2.3 promoter, to specifically drive osteoblast-specific gene expression in vivo. We subcloned the mouse Wnt4 gene into pGL647, flanked by the Col2.3 promoter. The fragments of the Wnt4 transgene were purified and microinjected into C57BL/6×SJL mouse oocytes (Charles River Laboratory), and the oocytes were surgically transferred to pseudopregnant C57BL/6 dams by the University of Michigan Transgenic Animal Model Core. We screened the founders by PCR using mouse tail genomic DNA and confirmed them by Southern blot analysis. We bred two transgenic founder mice with C57BL/6 mice for six generations to obtain a defined genetic background. TNFtg mice expressing hemizygous human TNF were purchased from Taconic Farms (#1006; B6.Cg(SJL)-Tg(TNF) N21+; Oxnard, Calif.). WT C57BL/6 mice for rWnt4 injection were purchased from Jackson Laboratory (Bar Harbor, Me.). In all experiments, female transgenic mice and female WT littermates as controls were used. We established a sample size of at least 8 mice per group in OVX and aging experiments based on our previous experience²². We used a sample size of at least 6 mice per group in TNFtg/Wnt4 experiments. The animals were randomly assigned to procedure groups including sham, OVX and rWnt4 injection. However, not all animal experiments were conducted in a completely blinded fashion. We ovariectomized 3-month-old transgenic and WT mice to induce osteoporosis. Two months after operation, we euthanized the mice and gathered their femurs for histological and μCT analysis. We collected blood samples and isolated serums for serology. Serum ELISAs were performed with a mouse Trap5b assay kit (SBA Sciences), an Ocn ELISA kit (Biomedical Technologies), Il-6 and Tnf Quantikine ELISA kits (R&D Systems). All mouse protocols were approved by The University Committee on Use and Care of Animals at the University of Michigan, the Animal Research Committee at the University of California, Los Angeles, or both.

### Cell Culture and Viral Infection.

We grew cells in a humidified 5% CO₂ incubator at 37° C. in alpha modified Eagle's medium supplemented with 15% FBS (FBS; Invitrogen, California, USA). Viral packaging was prepared as described previously⁵⁹. For viral infection, we plated cells overnight and then infected them with lentiviruses or retroviruses in the presence of polybrene (6 μg ml⁻¹, Sigma-Aldrich, USA) for 6 h. We then selected the cells with puromycin for 3 d. Resistant clones were pooled and knockdown or overexpression was confirmed via western blot analysis. For culturing of RAW264.7 cells (ATCC, Virginia, USA), we used Dulbecco's modified Eagle's medium supplemented with 10% FBS. Cells were newly purchased but were not tested for mycoplasma infection. For primary bone marrow macrophages, we extracted bone marrow cells from mouse femurs and treated them with 100 ng ml⁻¹ mouse macrophage colony-stimulating factor (M-Csf; R&D systems) for 2 d. This allowed the induction to form osteoclast precursors used in the experiments. For induction of osteoclastogenesis, we treated the osteoclast precursors with 100 ng ml⁻¹ mouse Rankl (R&D systems) for up to 3 d. In all in vitro experiments involving Wnt3a and Wnt4 recombinant proteins (R&D systems) and Rankl, we used 100 ng ml⁻¹.

### Western Blot Analysis.

We lysed cells in RIPA buffer (10 mM Tris-HCl, 1 mM EDTA, 1% sodium dodecyl sulfate (SDS), 1% Nonidet P-40, 1:100 proteinase inhibitor cocktail, 50 mM β-glycerophosphate, 50 mM sodium fluoride). We then separated lysates on a 10% SDS polyacrylamide gel and transferred to membranes by a semidry transfer apparatus (Bio-Rad). We blocked membranes with 5% milk for 1 h and then incubated with primary antibodies overnight. After rinsing, we incubated the immunocomplexes with horseradish peroxidase-conjugated anti-rabbit or anti-mouse IgG (Promega, Madison, Wis.) and visualized the membranes with SuperSignal Chemiluminiscent substrate (Pierce, Rockford, Ill.) as previously described^{22,59}. We used the following primary antibodies: anti-phospho-Tak1 (1:1,000; 4531S; Cell Signaling, Danvers, Mass.), anti-Tak1 (1:1,000; MAB5307; R&D systems), anti-phospho-p65 (1:2,000; 3033S; Cell Signaling), anti-p65 (1:2,000; 06-418; Millipore, Billerica, Mass.), anti-phospho-IκBα (1:1,000; 9246; Cell Signaling), anti-IκBα (1:1,000; sc-371; Santa Cruz, Santa Cruz, Calif.), anti-phospho-JNK (1:500; 9251; Cell Signaling), anti-JNK (1:1,000; 9258; Cell Signaling), anti-phospho-p38 (1:1,000; 9215; Cell Signaling), anti-p38 (1:1,000; 8680; Cell Signaling), anti-phospho-Erk (1:1,000; 4284; Cell Signaling), anti-Erk (1:1,000; 4696; Cell Signaling), anti-Traf6 (2 μg for immunoprecipitation; 1:1,000 for western blot; sc-8409; Santa Cruz), anti-Nlk (2 μg for immunoprecipitation; AB10206, Millipore), anti-Tab2 (1:1,000; 3744; Cell Signaling), anti-Nfatc1 (1:1,000; sc-7294; Santa Cruz), anti-HA (1:2,000; H9658; Sigma-Aldrich), anti-Tbp (1:2,000; T1827; Sigma-Aldrich) and anti-α-tubulin (1:10,000; 75168; Sigma-Aldrich).

### Alkaline Phosphatase, Alizarin Red and TRAP Staining.

To induce MSC differentiation, we cultured MSCs in mineralization-inducing medium containing 100 μM ascorbic acid, 2 mM β-glycerophosphate and 10 nM dexamethasone. For ALP staining, after induction, we fixed cells with 4% paraformaldehyde and incubated them with a solution of 0.25% naphthol AS-BI phosphate and 0.75% Fast Blue BB dissolved in 0.1 M Tris buffer (pH 9.3). For detecting mineralization, we induced MSCs for 2-3 weeks, fixed the cells with 4% paraformaldehyde and stained them with 2% Alizarin red solution (Sigma-Aldrich). To perform TRAP staining and osteoclast quantification, we fixed cells with a mixture of 3% formaldehyde, 67% acetone and 25% citrate solution and then stained with a TRAP kit from Sigma Aldrich according to manufacturers' instructions. Images were taken and analyzed using an Olympus IX-51 microscope. We only counted TRAP⁺ multinucleated cells (>3 nuclei) as osteoclasts.

### Luciferase Assays.

We infected primary bone marrow macrophages with lentiviruses expressing NF-κB-dependent or TOPflash luciferase reporters (System Biosciences) for 48 h simultaneously with M-CSF treatment. After stimulation with Rankl or Wnt3a or Wnt4 for 16 h, we isolated cell lysates. We then used a Dual-luciferase Reporter Assay System to measure luciferase activities as described previously⁵⁹.

### Real-Time RT-PCR and Chromatin Immunoprecipitation Assays.

We isolated total RNA from MSCs using Trizol reagents (Invitrogen). 2-μg aliquots of RNAs were synthesized using random hexamers and reverse transcriptase according to the manufacturer's protocol (Invitrogen). We then performed real-time PCR reactions using the QuantiTect SYBR Green PCR kit (Qiagen) and the Icycler iQ Multi-color Real-time PCR Detection System. The primers for 18S rRNA are: forward, 5′-CGGCTACCAC ATCCAAGGAA-3′; reverse, 5′-GCTGGAATTACCGCGGCT-3′. The primers for Runx2 are: forward, 5′-AGGGACTATGGCGTCAAACA-3′; reverse, 5′-GGCTCACGTCGCTCACTT-3′. The primers for Sp7 are: forward, 5′-CGCTTTGTGCCTTTGAAAT-3′; reverse, 5′-CCGTCAACGACGTTATGC-3′. The primers for Bglap are: forward, 5′-AGCAAAGGTGCAGCCTTTGT-3′; reverse, 5′-GCGCCTGGGTCTCTTCACT-3′. The primers for Alp are: forward, 5′-GGACAGGACACACACACACA-3′; reverse, 5′-CAAACAGGAGAGCCACTTCA-3′. The primers for Ibsp are: forward, 5′-ACAATCCGTGCCACTCACT-3′; reverse, 5′-TTTCATCGAGAAAGCACAGG-3′. The primers for Acp5 are: forward, 5′-GTGCTGCTGGGCCTACAAAT-3′; reverse, 5′-TTCTGGCGATCTCTTTGGCAT-3′. The primers for Mmp9 are: forward, 5′-TCCTTGCAATGTGGATGT-3′; reverse, 5′-CTTCCAGTACCAACCGTCCT-3′. The primers for Ctsk are: forward, 5′-GAAGAAGACTCACCAGAAGCAG-3′; reverse, 5′-TCCAGGTTATGGGCAGAGATT-3′. The primers for Birc3 are: forward, 5′-ACGCAGCAATCGTGCATTTTG-3′; reverse, 5′-CCTATAACGAGGTCACTGACGG-3′. The primers for Ptgs2 are: forward, 5′-AACCCAGGGGATCGAGTGT-3′; reverse, 5′-CGCAGCTCAGTGTTTGGG-3′. The primers for Tnf are: forward, 5′-CTGTAGCCCACGTCGTAGC-3′; reverse, 5′-TTGAGATCCATGCCGTTG-3′. The primers for Dkk1 are: forward, 5′-CTCATCAATTCCAACGCGATCA-3′; reverse, 5′-GCCCTCATAGAGAACTCCCG-3′. The primers for Axin2 are: forward, 5′-TGACTCTCCTTCCAGATCCCA-3′; reverse, 5′-TGCCCACACTAGGCTGACA-3′. The primers for Wnt4 (endogenous) are: forward, 5′-CTGGAGAAGTGTGGCTGTGA-3′; reverse, 5′-CAGCCTCGTTGTTGTGAAGA-3′. The primers for Opg are: forward, 5′-ACCCAGAAACTGGTCATCAGC-3′; reverse, 5′-CTGCAATACACACACTCATCACT-3′. The primers for Tnfsfl1 are: forward, 5′-CAGCTATGATGGAAGGCTCA-3′; reverse, 5′-GACTTTATGGAACCCGA-3′.

For extraction of tissue RNA, we dissected mouse femurs and pulverized them in liquid nitrogen. After extracting total RNA as described above, we removed residual genomic DNA using Turbo DNA-free DNase removal kit (Ambion). For RT-PCR, primers specific for Wnt4 transgene are: forward, 5′-CTAAAGCCATTGACGGCTGC-3′; reverse, 5′-GCGTAATCTGGAACATCATATGGG-3′. Primers for β-actin are: forward, 5′-CGTCTTCCCCTCCATCG-3′; reverse, 5′-CTCGTTAATGTCACGCAC-3′.

We performed ChIP assays using a ChIP assay kit (Upstate, USA) following the manufacturer's recommendation. Briefly, we incubated cells with a dimethyl 3,3′ dithiobispropionimidate-HCl (Pierce) solution (5 mM) for 10 min at room temperature, followed by formaldehyde treatment for 15 min in a 37° C. water bath. For each ChIP reaction, we used 2×10⁶ cells. We then quantified resulting precipitated DNA samples with real-time PCR and expressed data as the percentage of input DNA. Antibodies for ChIP assays were purchased from the following commercial sources: polyclonal anti-p65 (Millipore); polyclonal anti-NFATc1 (Santa Cruz). The primers for Nfatc1 are: forward, 5′-CTGTGTTCCCACATGTCCTC-3′; reverse, 5′-GCGACTGCAGTGTGTTCTTT-3′. 9 kb downstream for Nfatc1 are: forward, 5′-CTGGCACCAAAGTTGAGAGA-3′; reverse, 5′-GATGGCTCTACCTGCACAGA-3′.

### OVX, Bone Histomorphometry and Scoring of Arthritic Joint Swelling.

We performed OVX or sham operation on 3-month-old female WT and Wnt4 mice under isofluorane anesthesia. For the preventive model, rWnt4 protein (8 μg kg⁻¹) were intraperitoneally injected daily for 3 weeks immediately after the surgery. For dual labeling, mice received intraperitoneal injection of calcein (0.5 mg per mouse, Sigma-Aldrich) 10 and 3 d before euthanasia. Mice were euthanized 1 month after OVX. For the therapeutic model, we first performed OVX on 3-month-old mice and waited for 1 month to establish bone loss. Mice received intraperitoneal injection of rWnt4 (20 μg kg⁻¹) or vehicle control daily for 1 month before collection of bone samples. Eight to twelve mice were used in each group.

Following euthanasia, we fixed right femurs in 70% ethanol for 48 h and embedded in methyl methacrylate. 8-μm longitudinal sections were either stained with toluidine blue for osteoblast count or examined under fluorescent microscope to evaluate BFR and MAR as described previously²². We fixed left femurs in 10% formaldehyde and embedded them in paraffin for preparation in 5-μm-thick sections. We analyzed osteoclast parameters after TRAP staining as described. For all brightfield and fluorescent microscopy analysis, we used Olympus-IX51 inverted microscope with SPOT advanced 4.0 and CellSens software.

We scored the swelling of hindpaws on 1-year-old TNFtg and TNFtg/Wnt4 mice on a scale of 0 to 3, as previously described^{60,61}: 1=mild arthritis (mild swelling of joint and paw); 2=moderate arthritis (severe swelling and joint deviation); and 3=severe arthritis (ankylosis detected upon flexion). We used histological sections of hindpaw and ankle joints to examine the tibiotalar and interdigital joints and performed μCT imaging to further evaluate bone erosion and destruction of joint space associated with arthritis.

### Immunostaining and μCT Analysis of Mice.

We extracted femurs from euthanized mice and fixed them in 10% neutral buffered formalin for 24 h. For μCT scanning, the specimens were fitted in a cylindrical sample holder (20.5 mm in diameter) with the long axis of the femur perpendicular to the X-ray source. We used a Scanco μCT40 scanner (Scanco Medical) set to 55 kVp and 70 μA. The bone volume (mm³) over tissue volume and bone mineral density in the region of interest were measured directly with μCT Evaluation Program V4.4A (Scanco Medical). We defined the regions of interest as the areas between 0.3 mm and 0.4 mm proximal to the growth plate in the distal femurs in order to include the secondary trabecular spongiosa. A threshold of 250 was used for evaluation of all scans²². For visualization, we imported the segmented data and reconstructed them as a three-dimensional image displayed in μCT Ray V3.0 (Scanco Medical).

After scanning, we decalcified the specimens and sectioned them for staining as previously described²². Antibodies used included rabbit polyclonal anti-NLS-p65 (600-401-271; 1:200; Rockland), rabbit polyclonal anti-Mmp9 (38898; 1:500; Abcam), rabbit polyclonal anti-Tnf (34674, 1:200; Abcam), and rabbit polyclonal anti-Cox2 (15191, 1:400, Abcam). For quantification of p65 positive staining, we selected at least ten images from each section per femur, measured the integral optical density (IOD) of nuclear-stained p65 using the Image Pro Plus 6.0 software (MediaCybernetics). We normalized the IOD by stained area and presented the data as reported previously⁶².

### Statistical Analyses.

Biostatistic analyses were performed with the assistance of Dr. Gang Li at the Biostatistic Core of the UCLA Jonsson Comprehensive Cancer Center. Numerical data and histograms were expressed as the mean±s.d. Statistical analyses were performed on data distributed in normal pattern. When single comparisons were made between two groups, two-tailed Student's t-test was performed. When multiple comparisons were made across treatment groups in cases of OVX, TNFtg/Wnt4 and Wnt4 injection experiments, one-way analysis of variance (ANOVA) with Tukey's post hoc test was performed to account for type-I errors. A difference was considered statistically significant with P<0.05. To assess if the rate of decline was statistically significant between OB-Wnt4 and WT mice, two-way ANOVA was employed to examine the interaction between age and genotype. An interaction with P<0.05 was considered statistically significant.

### Results

### Wnt4 Promotes Bone Formation In Vivo

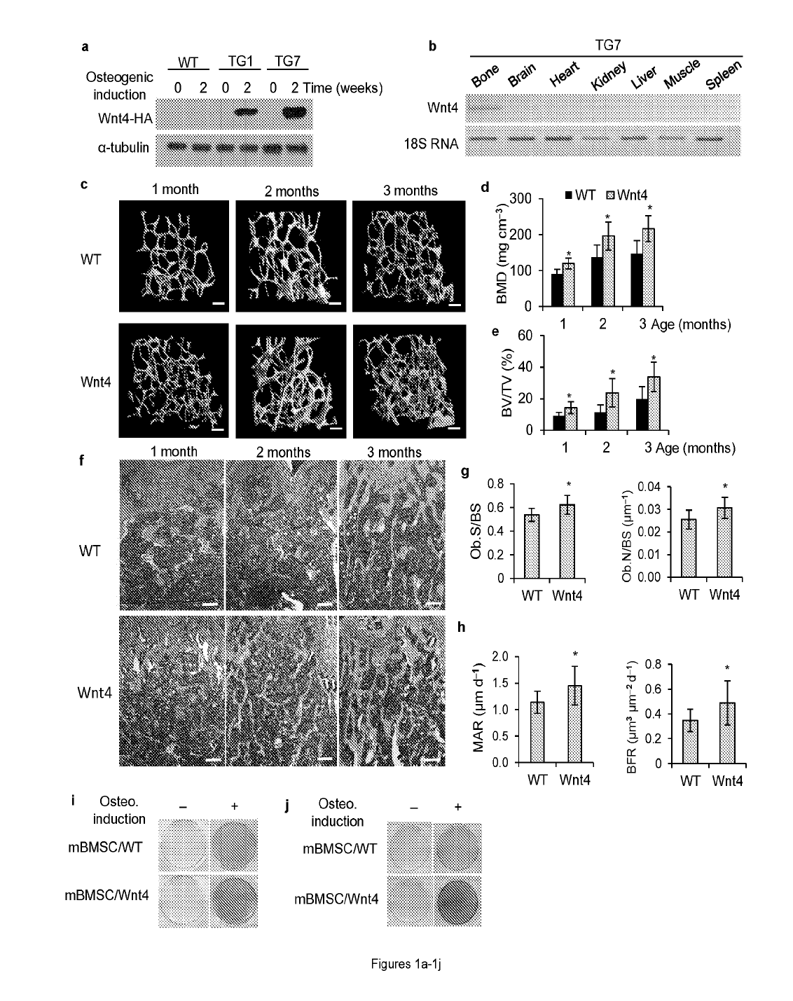

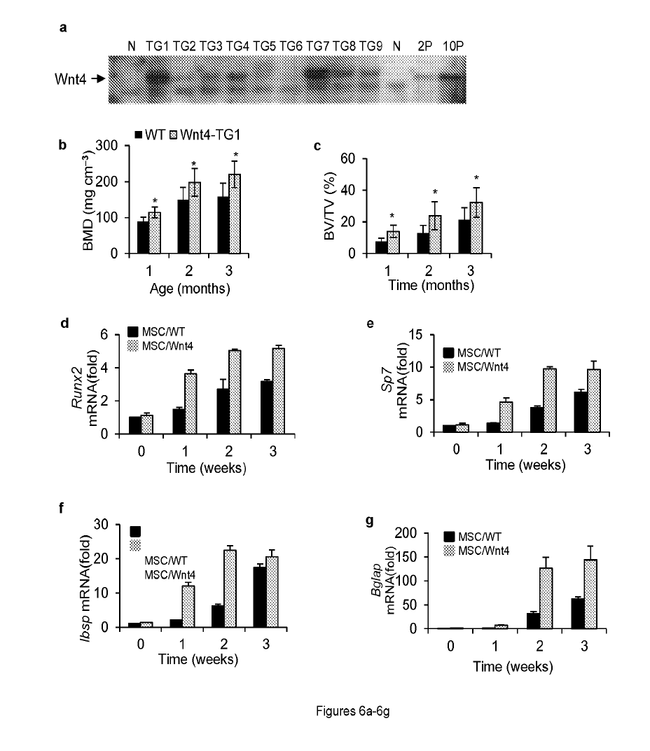

To explore whether Wnt4 promoted bone formation in vivo, we generated transgenic mice in which Wnt4 is driven by the mouse 2.3-kb type 1 collagen (Col2.3) promoter (OB-Wnt4 mice). The Col2.3 promoter contains a 2.3-kb DNA fragment upstream of the transcription start site of the Col1a1 gene³⁴ and has been shown to drive gene expression specifically in differentiated osteoblasts³⁵. The fragments of the Wnt4 transgene were microinjected into C57BL/6×SJL mouse oocytes, and the oocytes were surgically transferred to pseudopregnant C57BL/6 dams. Seven of the ten potential founders screened displayed strong expression of the transgene (FIG. 6a), thus allowing establishment of two separate transgenic mouse lines (TG1 and TG7). Whereas hemaglutinin-tagged transgenic Wnt4 was undetectable in primary calvarial cells from wild-type (WT) C57BL/6 mice, Wnt4 in calvarial cells from TG1 and TG7 mouse lines were induced as the cells differentiated into osteoblasts in osteogenic medium (FIG. 1a). RT-PCR confirmed that Wnt4 transgene mRNA was expressed in bone tissue but not in brain, heart, kidney, liver, spleen or muscle using the TG7 line (FIG. 1b).

Next, we investigated whether Wnt4 could enhance bone formation in vivo using the TG7 line. Of note, both lines of OB-Wnt4 mice had phenotypically normal skeleton at birth (data not shown). Microcomputed tomography (XT) analysis of the secondary spongiosa of the distal femur metaphysis revealed that the bone mineral density (BMD) of Wnt4 mice was significantly higher compared to WT littermates at 1, 2 and 3 moFigurenths of age (FIGS. 1c,d). Similarly, the bone volume/tissue volume ratio (BV/TV) was significantly higher in OB-Wnt4 mice compared to WT mice (FIG. 1e), consistent with the greater amount of trabecular bones shown in H&E staining (FIG. 1f). Histomorphometric analysis revealed mildly higher osteoblast counts in 3-month-old OB-Wnt4 mice compared to WT mice (FIG. 1g). To further confirm whether the increased BMD was due to enhanced osteoblast function, we performed dynamic histomorphometric analysis over a 7-d period using calcein labeling²² and found that the bone formation rate (BFR) in 3-month-old OB-Wnt4 mice was significantly higher compared to WT mice (FIG. 1h). Similarly, characterization of the TG1 mouse line also confirmed that Wnt4 enhanced bone formation in vivo, ruling out variations due to mouse strains (FIGS. 6b,c).

To examine whether Wnt4 enhanced osteoblastic activity in a cell-autonomous manner, we isolated bone marrow MSCs from femurs of OB-Wnt4 mice and WT mice. Primary MSCs from Wnt4 mice demonstrated enhanced osteogenic potential, as evidenced by alkaline phosphatase and Alizarin red staining, when cells were induced by osteogenic medium (FIGS. 1i,j). Furthermore, real-time RT-PCR showed greater mRNA expression of the master osteogenic transcription factors Runx2 and Sp7 (FIGS. 6d,e), as well as the mineralization markers Ibsp and Bglap (FIGS. 6f,g), in differentiated osteoblasts from Wnt4 mice compared with WT mice.

### Wnt4 Prevents Estrogen Deficiency-Induced Bone Loss

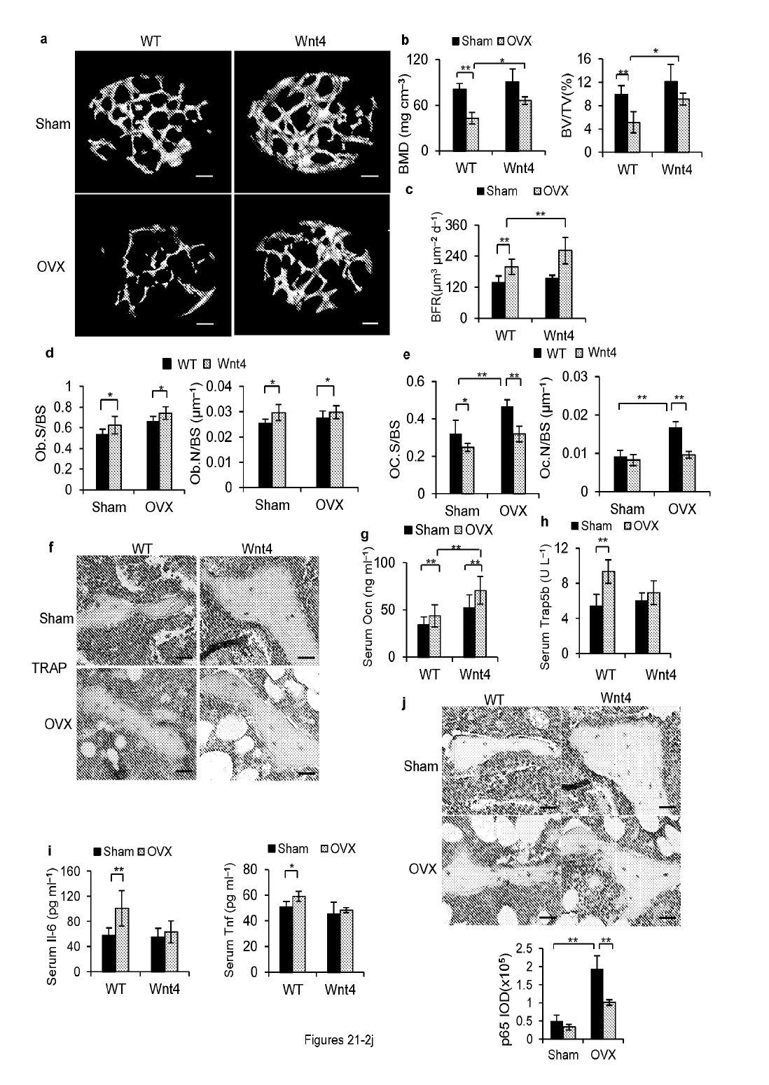

To mimic the molecular pathogenesis of osteoporotic bone loss, mouse ovariectomy (OVX) has been widely used as an animal model of this condition^{6,22,36}. We performed OVX or sham operation on 3-month-old WT and OB-Wnt4 mice, followed by μCT analysis of femurs from these mice. We found noticeable trabecular bone loss in WT mice compared to sham controls 2 months after OVX. In contrast, bone loss was markedly lower in OB-Wnt4 mice after OVX compared to WT mice (FIG. 2a). Quantitative measurements showed that whereas 47% of BMD and 48% of BV/TV were lost in WT mice after OVX, only 27% of BMD and 24% of BV/TV were lost in OB-Wnt4 mice (FIG. 2b). Following OVX, BFR was higher in WT mice to compensate for the accelerated bone resorption, whereas in OB-Wnt4 mice, osteoblastic activity was further enhanced (FIG. 2c). Similarly, toluidine blue staining revealed significantly higher osteoblast number osteoblast surface in OB-Wnt4 as compared to WT mice in both OVX and sham groups (FIG. 2d). In contrast, we observed lower osteoclast number and surface in OB-Wnt4 mice compared to WT mice in both OVX and sham groups (FIGS. 2e,f). We also performed ELISA to assess the serum markers of bone turnover. The serum concentrations of osteocalcin, a marker of bone formation, were significantly higher in OB-Wnt4 mice compared to WT mice after OVX (FIG. 2g). In contrast, OVX induced higher serum concentrations of Trap5b, a marker of bone resorption³⁷, in WT but not in OB-Wnt4 mice (FIG. 2h).

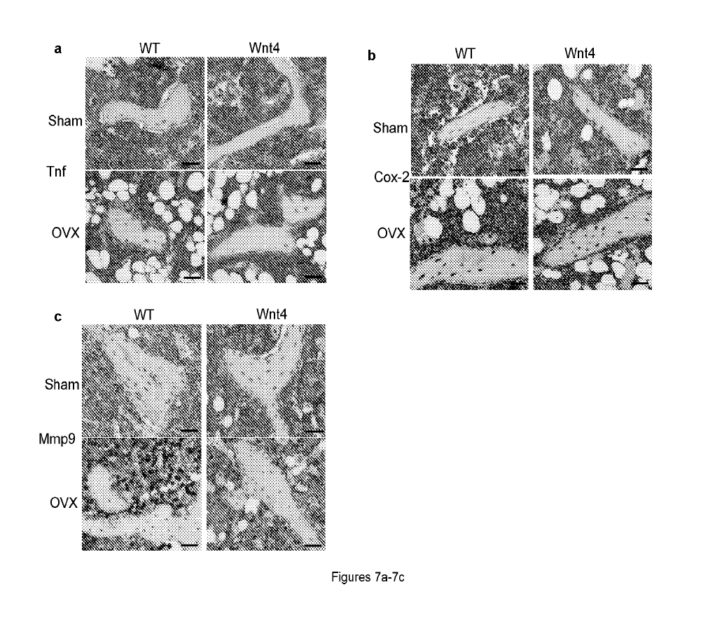

Studies have implicated proinflammatory cytokines, including tumor necrosis factor (Tnf) and interleukin-6 (Il-6), as important mediators of accelerated bone loss in osteoporosis^{38,39}. Consistent with this, OVX induced an elevation in serum concentrations of Tnf and Il-6 in WT mice, but such induction was suppressed in OB-Wnt4 mice (FIG. 2i). Immunostaining of activated p65 in femur sections revealed enhanced NF-κB activity in osteoclasts and bone marrow cells surrounding trabecular bones following OVX. In contrast, the NF-κB activation by OVX was significantly less pronounced in OB-Wnt4 mice (FIG. 2j). To further confirm the inhibition of NF-κB by Wnt4 in vivo, we immunostained NF-κB-dependent targets, including Tnf, cycloxygenase-2 (Cox-2) and matrix metallopeptidase-9 (Mmp9). Consistent with activated p65 staining, we found that Wnt4 also potently reduced the expression of Tnf, Cox-2 and Mmp9 induced by OVX in vivo (FIGS. 7a-c).

### Wnt4 Inhibits TNF-Induced Inflammatory Bone Loss

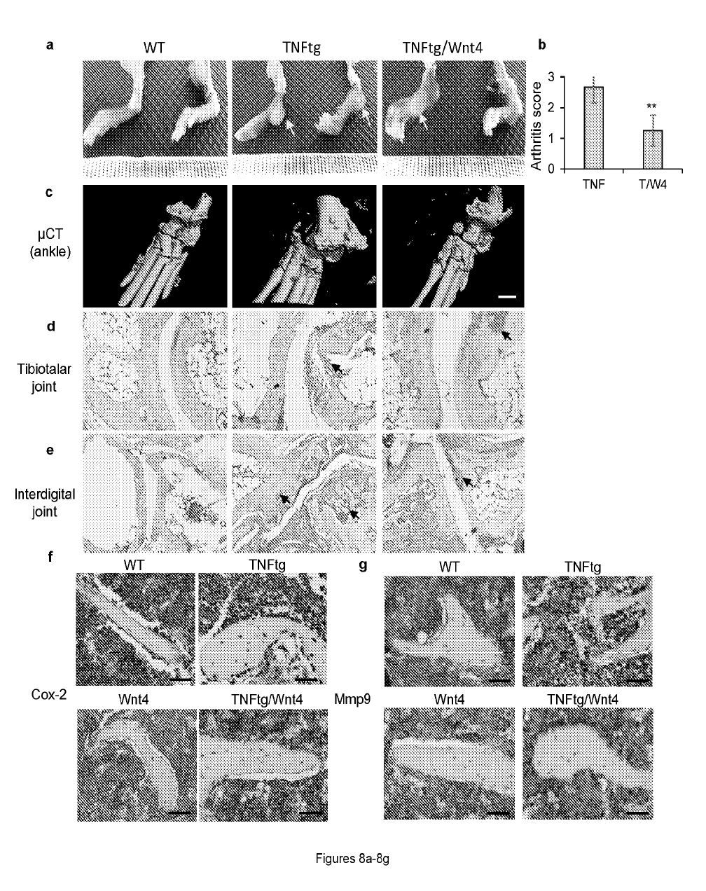

TNF potently induces inflammation by activating NF-κB. Transgenic mice overexpressing human TNF (TNFtg) develop systemic bone loss and osteoporosis in addition to erosive arthritis due to a higher degree of osteoclastogenesis and inhibition of bone formation⁴⁰⁻⁴². To further determine whether Wnt4 could directly inhibit inflammation-associated bone loss, we bred TNFtg mice with OB-Wnt4 mice (TNFtg/OB-Wnt4 mice). There was severe paw and joint swelling, often associated with joint deviation, in 1-year-old TNFtg mice. We also performed μCT and histological analyses, which revealed extensive joint cartilage destruction and bone erosions due to invasion of inflammatory cells (FIGS. 8a-e). In contrast, there was significantly less joint swelling, bone erosion and inflammation in TNFtg/OB-Wnt4 mice than in TNFtg mice of comparable age (FIGS. 8a-e).

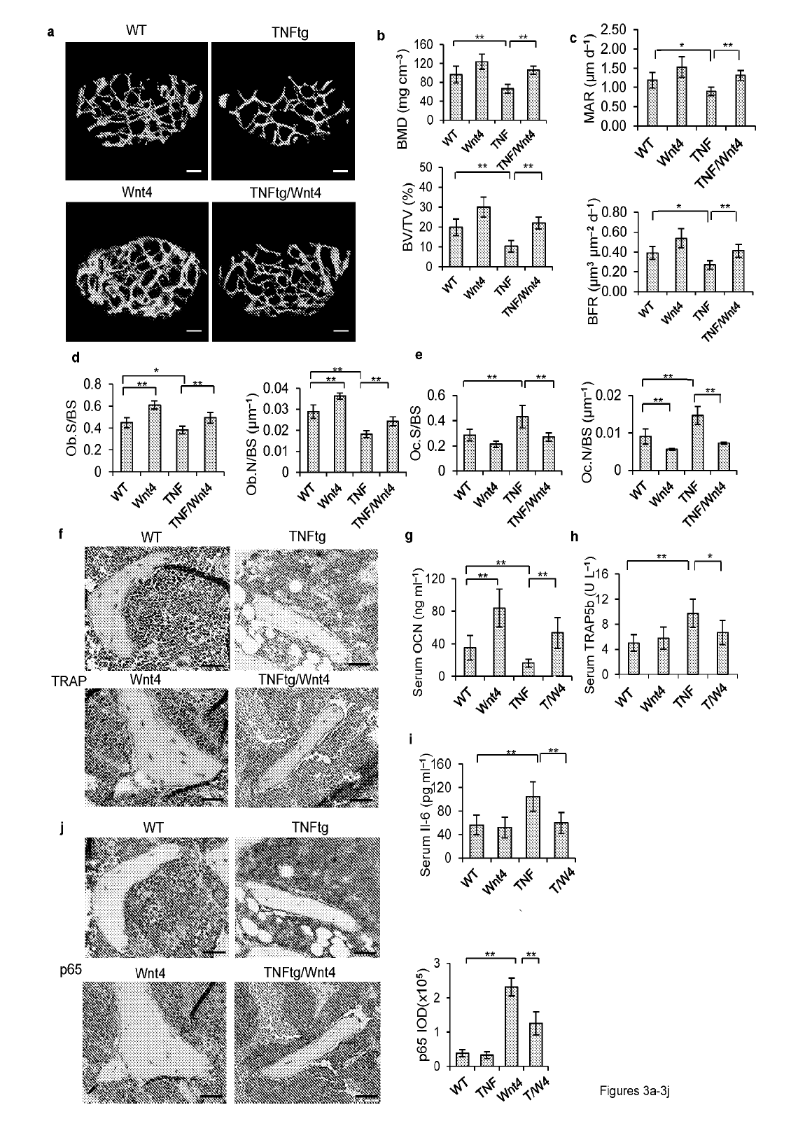

Consistent with previous studies, μCT analysis revealed systemic bone loss suffered by 1-year-old TNFtg mice compared with WT mice. However, bone loss in 1-year-old TNFtg/OB-Wnt4 femurs was markedly mitigated (FIG. 3a). Quantitative measurements revealed that whereas 31% of BMD and 68% of BV/TV were lost in TNFtg mice compared to WT mice, only 18% of BMD and 28% of BV/TV were lost in TNFtg/OB-Wnt4 mice (FIG. 3b). As the reduced bone loss could be due to either higher bone formation or lower bone resorption (or both), we examined the effect of Wnt4 on both components of bone homeostasis. The lower degree of BFR and mineral apposition rate (MAR) in TNFtg compared to WT mice were alleviated in TNFtg/OB-Wnt4 mice (FIG. 3c). Consistent with this finding, histomorphometric analysis also showed 24% greater osteoblast counts in TNFtg/OB-Wnt4 than in TNFtg mice (FIG. 3d).

As it has been shown that osteoclastogenesis and bone resorption were enhanced in TNFtg mice⁴⁰⁻⁴², we next examined the effect of Wnt4 on accelerated bone resorption in TNFtg mice. Both histomorphometric analysis and tartrate-resistant acid phosphatase (TRAP) staining revealed that whereas osteoclast activity was higher in TNFtg mice compared to WT controls, it was significantly lower in TNFtg/OB-Wnt4 mice (FIGS. 3e,f). Consistent with this, the serum concentrations of osteocalcin were significantly lower in TNFtg mice than in TNFtg/OB-Wnt4 mice (FIG. 3g). On the other hand, the serum concentrations of Trap5b were significantly higher in TNFtg mice than in TNFtg/OB-Wnt4 mice (FIG. 3h).

We observed that the serum Il-6 concentration in TNFtg/Wnt4 mice was only 55% of that in TNFtg mice (FIG. 3i). As TNF is a potent activator of NF-κB signaling, which is associated with osteoporosis^{16,22,43}, we next examined whether Wnt4 might inhibit TNF-induced NF-κB activation in the TNFtg/OB-Wnt4 mice. Immunostaining of active p65 revealed markedly enhanced NF-κB activity in the proximity of trabecular bones in TNFtg mice, whereas NF-κB staining was significantly reduced in TNFtg/OB-Wnt4 mice (FIG. 3j). Moreover, Wnt4 also inhibited the expression of Cox-2 and Mmp9 in osteoclasts and bone marrow cells induced by TNF in vivo (FIGS. 8f,g).

### Wnt4 Inhibits Tak1-NF-κB Signaling

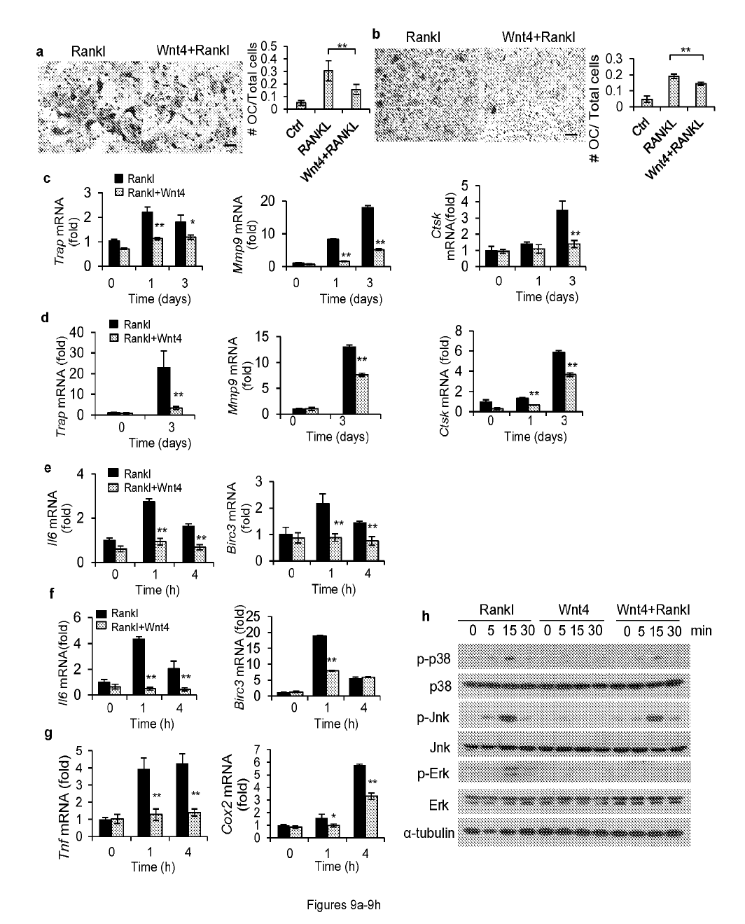

Our in vivo results suggest that Wnt4 secreted by osteoblasts may inhibit osteoclast formation and bone resorption in a paracrine fashion. To confirm our hypothesis, we examined whether Wnt4 could directly inhibit osteoclast differentiation using recombinant Wnt4 (rWnt4) protein. As evidenced by TRAP staining, rWnt4 protein significantly inhibited osteoclast differentiation of primary bone marrow macrophages induced by receptor activator of NF-κB ligand (Rankl; FIG. 9a). Similarly, the osteoclast-like differentiation of RAW264.7 cells induced by Rankl was also attenuated by Wnt4 (FIG. 9b). Real-time RT-PCR confirmed that rWnt4 inhibited the expression of osteoclast marker genes, including Acp5, Mmp9 and Ctsk, induced by Rankl in bone marrow macrophages and RAW264.7 cells (FIGS. 9c,d). As Wnt4 inhibited the expression of NF-κB target genes in vivo, we also examined whether rWnt4 inhibited the expression of NF-κB target genes induced by Rankl Real-time RT-PCR revealed that rWnt4 potently inhibited induction of the NF-κB-dependent genes Il6 and Birc3 by Rankl in bone marrow macrophages (FIG. 9e) and in RAW264.7 cells (FIG. 90. Consistent with our findings from immunostaining in vivo, rWnt4 also significantly suppressed the NF-κB-dependent genes Tnf and Ptgs2 in bone marrow macrophages (FIG. 9g).

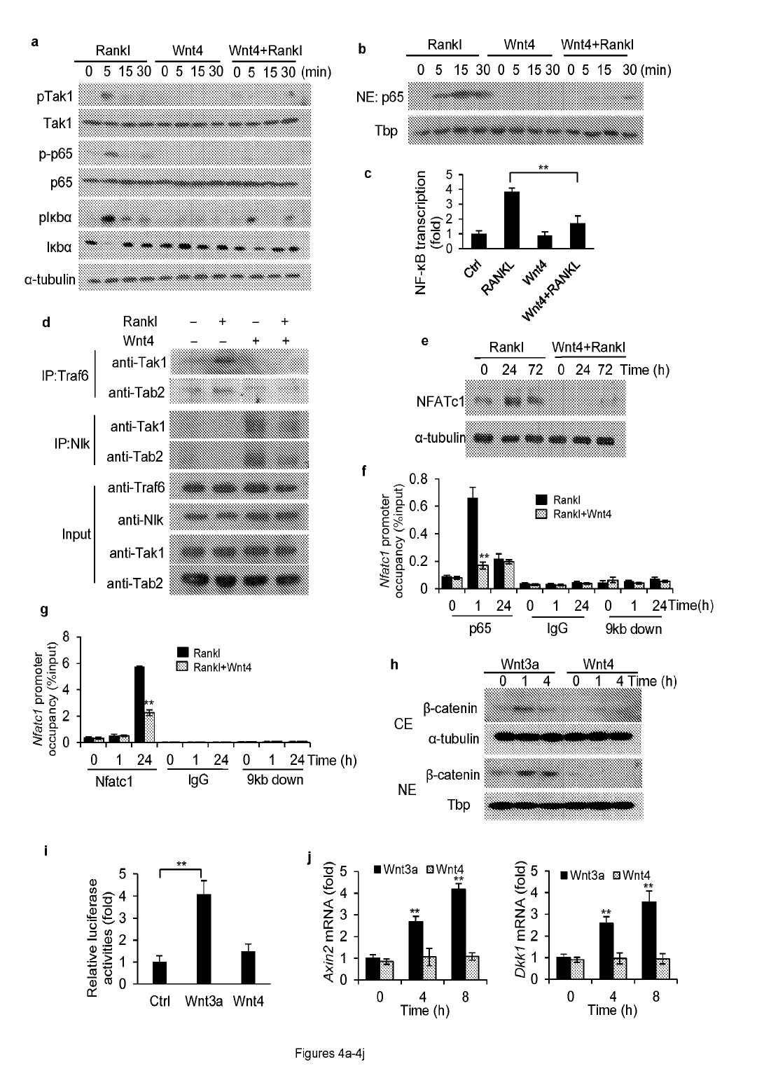

To further elucidate the molecular mechanism by which Wnt4 inhibited NF-κB and osteoclastogenesis, we examined each key step of NF-κB activation induced by Rankl Activation of the Rank receptor leads to association of its cytoplasmic domain with Tnf receptor-associated factor-6 (Traf6), which is essential in osteoclast differentiation^{47,48}. Traf6 forms a complex with Tak1 and Tak1-binding protein-2 (Tab2), leading to phosphorylation and activation of Tak1 (ref 49). In canonical NF-κB signaling, Tak1 then phosphorylates IκB kinase (IKK) complex and thereby initiates degradation of IκBα, followed by phosphorylation and nuclear translocation of p65 to activate downstream target genes⁴⁹. Western blot analysis revealed that rWnt4 potently inhibited Tak1 phosphorylation, as well as the subsequent phosphorylation of p65 and the phosphorylation and degradation of IκBα induced by Rankl (FIG. 4a). Furthermore, rWnt4 also suppressed Rankl-induced nuclear translocation of p65 (FIG. 4b). Moreover, rWnt4 inhibited NF-κB-dependent transcription, as determined by the NF-κB-dependent luciferase reporter assay (FIG. 4c).

Because Tak1 also forms a complex with Nemo-like kinase (Nlk) and Tab2 in noncanonical Wnt signaling^{27,50}, we hypothesized that rWnt4 stimulation may interfere with the formation of the Traf6-Tak1-Tab2 complex induced by Rankl Immunoprecipitation using antibodies specific to Traf6 revealed that Rankl induced the formation of the Traf6-Tak1-Tab2 complex (FIG. 4d). However, addition of rWnt4 drastically inhibited the formation of the Traf6-Tak1-Tab2 complex (FIG. 4d). In contrast, we observed that rWnt4 stimulation induced the formation of the Tak1-Tab2-Nlk complex, and the addition of Rankl partially reduced the formation of the Tak1-Tab2-Nlk complex (FIG. 4d). As Traf6-Tak1 signaling also activates p38 mitogen-activated protein kinase, Jnk and extracellular signal-regulated kinase (Erk), we examined whether rWnt4 inhibited the activation of p38, Jnk and Erk induced by Rankl We found that rWnt4 partially inhibited the phosphorylation of Erk, p38 and Jnk induced by Rankl FIG. 9h).

As the nuclear factor of associated T cells-c1 (Nfatc1) is the key transcription factor for osteoclastogenesis⁵¹, we examined the effect of rWnt4 treatment on its expression following Rankl stimulation in bone marrow macrophages. We found that the induction of Nfatc1 by Rankl was repressed by rWnt4 (FIG. 4e). Previously, it has been shown that activation of NF-κB induces expression of Nfatc1, which in turn activates osteoclast differentiation⁵². Both NF-κB and NFAT consensus binding sites exist at the Nfatc1 promoter. Upon induction by Rankl, p65 is recruited to the Nfatc1 promoter to activate its transcription, and subsequently, the newly generated Nfatc1 can autoamplify itself⁵². Chromatin immunoprecipitation (ChIP) assays revealed that rWnt4 significantly suppressed Rankl-induced p65 binding to the Nfatc1 promoter (FIG. 4f). Consequently, rWnt4 also potently reduced Nfatc1 binding at its own promoter induced by Rankl (FIG. 4g).

We previously found that Wnt4 activates noncanonical Wnt signaling in MSCs³³, but Wnt4 might also stimulate canonical Wnt signaling by stabilizing β-catenin. To rule out this possibility, we examined whether rWnt4 protein increased the levels of cytosolic and nuclear β-catenin in bone marrow macrophages. Subcellular fractionation revealed that whereas rWnt3a increased the levels of cytosolic and nuclear β-catenin, rWnt4 did not induce the accumulation of β-catenin (FIG. 4h). Moreover, we also examined whether rWnt4 induced β-catenin-dependent transcription using a TOPflash luciferase reporter. rWnt3a, but not rWnt4, significantly activated the luciferase reporter in bone marrow macrophages (FIG. 4i). In addition, two well-known Wnt-β-catenin pathway target genes, Axin2 and Dkk1, were induced by rWnt3a but not by rWnt4 (FIG. 4j).

rWnt4 Inhibits OVX-Induced Bone Loss

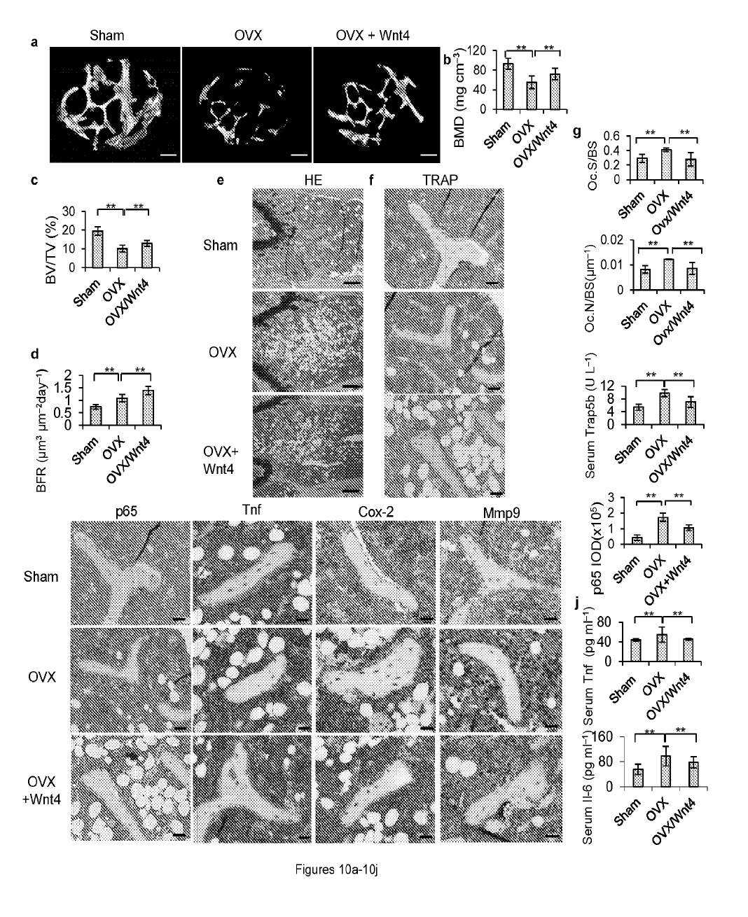

To explore whether Wnt4 can be used clinically, we first tested whether rWnt4 prevents bone loss by inhibiting NF-κB in an OVX mouse model. We ovariectomized 3-month-old mice and immediately began intraperitoneal administration of rWnt4 once a day for 3 weeks. We then performed μCT analysis, which showed that whereas mice that underwent OVX suffered marked loss in trabecular BMD and BV/TV 1 month after OVX, mice injected with rWnt4 had significantly less bone loss (FIGS. 10a-c). Histological analysis also confirmed that rWnt4 significantly inhibited trabecular bone loss induced by OVX (FIGS. 10e-g). Moreover, rWnt4 also reduced serum Trap5b levels (FIG. 10h). Immunostaining showed that rWnt4 inhibited NF-κB activity in osteoclasts and adjacent inflammatory cells upon OVX (FIG. 10i). Consistent with this, we found that serum levels of Il-6 and Tnf induced by OVX were significantly reduced by rWnt4 (FIG. 10j).

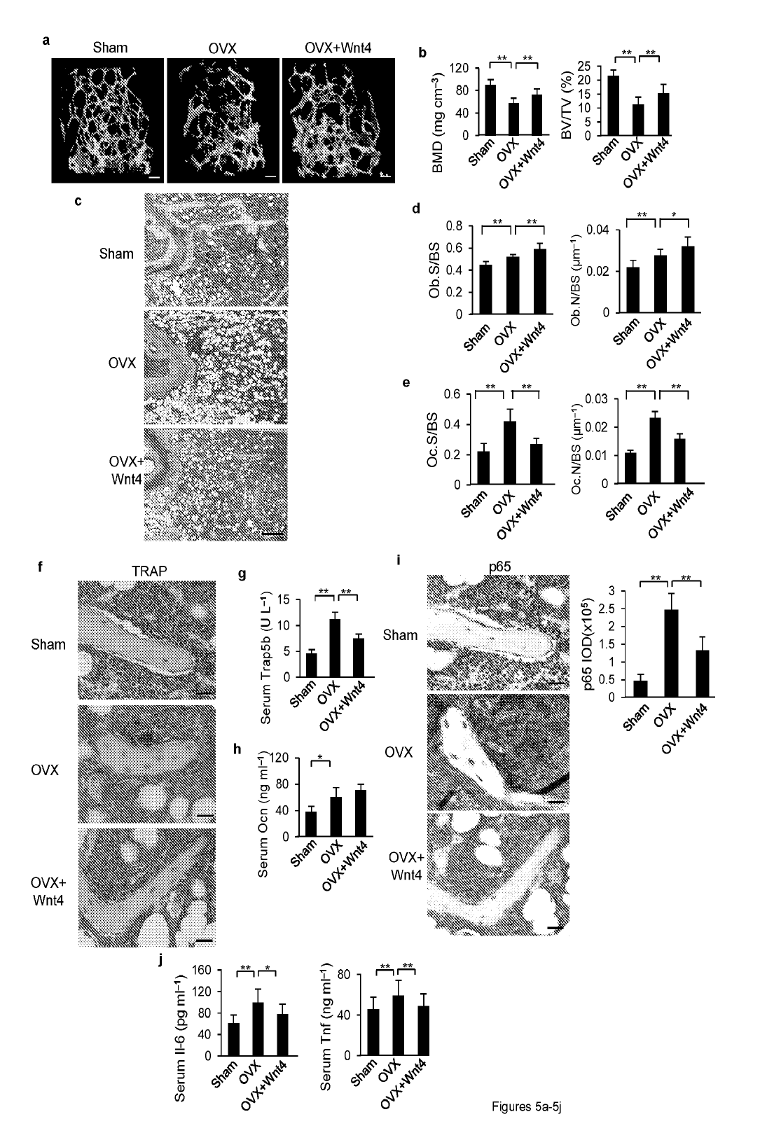

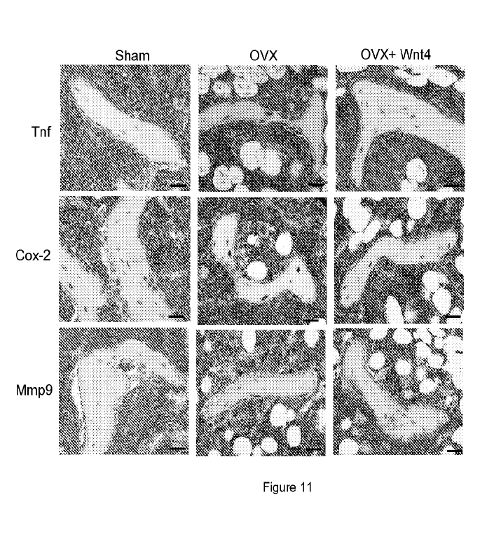

To further evaluate the therapeutic value of rWnt4 protein, we examined whether rWnt4 could reverse established bone loss in mice induced by OVX. We first performed OVX on 3-month-old mice and waited for 1 month to establish bone loss. We then administered rWnt4 or the vehicle control (PBS) to mice for 1 month. We performed μCT analysis and found that rWnt4 treatment in the OVX group was associated with significantly higher degrees of BMD and BV/TV compared to PBS-treated OVX mice (FIGS. 5a,b). Histological staining confirmed that rWnt4 treatment was associated with lower trabecular bone loss compared to PBS-treated OVX group (FIG. 5c). Histomorphometric analysis also showed that the rWnt4-treated OVX mice had significantly higher osteoblast counts and lower osteoclast numbers compared to PBS-treated OVX mice (FIGS. 5d-f). Consistent with these results, rWnt4 treatment of OVX mice was associated with lower serum Trap5b concentrations and higher serum osteocalcin concentrations versus those treated with PBS (FIGS. 5g,h). Immunostaining revealed that rWnt4 potently inhibited NF-□B activity in osteoclasts and adjacent bone marrow cells (Figure Si), as well as the expression of Tnf, Cox-2 and Mmp9 (FIG. 11). We found that rWnt4 treatment of OVX mice was also significantly associated with lower serum concentrations of Il-6 and Tnf than those treated with PBS (FIG. 5j).

### Discussions

We demonstrated that Wnt4 could reduce OVX- and inflammation-induced bone loss, and promote increased bone mass. Moreover, Wnt4 inhibited NF-κB activation induced by estrogen-deficiency and TNF, thus revealing a previously uncharacterized cross-talk between noncanonical Wnt signaling and NF-κB. Gain- or loss-of-function mutations of Wnt signaling components have been identified in a variety of human bone disorders^{26,53,54}. Recently, Wnt5a has been found to enhance osteoclast formation and bone resorption by activating the noncanonical Jnk signaling pathway. Wnt5a enhanced osteoclastogenesis induced by Rankl through the Ror2 receptor³², suggesting that targeting Wnt5a may prevent bone erosion in arthritis. However, Wnt5a-haploinsufficient mice had a bone-loss phenotype with increased adipogenesis in bone marrow⁵⁰. Thus, Wnt5a might not be an ideal therapeutic agent for arthritis and metabolic bone loss. On the contrary, we found that Wnt4 inhibited osteoclastogenesis and bone resorption in vitro and in vivo by inhibiting NF-κB while promoting bone formation, thereby holding more promise as a potential therapeutic agent for preventing skeletal aging, osteoporosis and arthritis compared to Wnt5a.

Various Wnt ligands can elicit different responses depending on their receptors and cell contexts. Wnt5a acts via Ror2 to enhance the expression of Rank in osteoclast precursors by stimulating activator protein-1 and promotes Rankl-induced osteoclast formation³². Notably, we found that Wnt4 suppresses Tak1 activation induced by Rankl, resulting in the inhibition of IKK-NF-κB signaling activation in macrophages and osteoclast precursors. Although Tak1 plays a role in noncanonical Wnt signaling by interacting with Nlk⁵⁰, it also modulates canonical Wnt signaling^{55,56}. The definitive role of Tak1 in both canonical and noncanonical signaling might depend on cell context and individual Wnt ligands. Our results suggest that Wnt4 might activate its receptors to promote Tak1-mediated noncanonical Wnt signaling in osteoclasts and subsequently sequester Tak1 from effectively binding with Traf6 to induce the NF-κB signaling cascade. Although we showed that Wnt4 promoted Tak1 binding to Nlk, it is possible that Wnt4 might also promote the interaction between Tak1 and the Wnt signaling components, as it has been reported that Ror2 interacts with Tak1⁵⁵.

Most drugs currently used for osteoporosis are inhibitors of bone resorption, but they cannot restore the substantial bone loss that has already occurred at the time of diagnosis. Therefore, a better treatment module for osteoporosis would not only block bone catabolism but also promote bone anabolism while controlling local inflammation^{6-8,11}. Multiple Wnt proteins, including Wnt4, have been detected in bone tissues or bone marrow^{54,57,58}. Although the inhibition of osteoporotic bone loss and inflammation is mainly based on transgenic overexpression of Wnt4, multiple noncanonical Wnt ligands, including Wnt4, Wnt6, Wnt11 and Wnt16, are expressed in osteoprogenitors^{57,58}. They may collectively protect against aging-associated bone loss and inflammation. Notably, we show that rWnt4 protein effectively inhibit OVX-induced bone loss by inhibiting NF-κB. Although canonical Wnt proteins have potential therapeutic value for treating osteoporosis by promoting bone formation, the constitutive activation of β-catenin might also increase the risk for cancer development that is associated with aging^{23,24}. As Wnt4 does not activate β-catenin in either osteoblasts or osteoclasts, and by inhibiting NF-κB, our results suggest that rWnt4 may be a better, and perhaps safer, therapeutic agent for preventing osteoporosis and treating inflammatory bone diseases.

### REFERENCES

1. Manolagas, S. C. & Jilka, R. L. Bone marrow, cytokines, and bone remodeling. Emerging insights into the pathophysiology of osteoporosis. N. Engl. J. Med. 332, 305-311 (1995).
2. Zaidi, M. Skeletal remodeling in health and disease. Nat. Med. 13, 791-801 (2007).
3. Manolagas, S. C. & Parfitt, A. M. What old means to bone. Trends Endocrinol Metab. 21, 369-374 (2010).
4. Chien, K. R. & Karsenty, G. Longevity and lineages: toward the integrative biology of degenerative diseases in heart, muscle, and bone. Cell 120, 533-544 (2005).
5. Bruunsgaard, H. et al. Predicting death from tumour necrosis factor-alpha and interleukin-6 in 80-year-old people. Clin Exp Immunol 132, 24-31 (2003).
6. Weitzmann, M. N. & Pacifici, R. Estrogen deficiency and bone loss: an inflammatory tale. J Clin. Inv. 116, 1186-1194 (2006).
7. McClean, R. Proinflammatory cytokines and osteoporosis. Curr Osteoporos Rep. 7, 134-139 (2009).
8. Lopez-Otin, C., Blasco, M. A., Partridge, L., Serrano, M., & Kroemer, G. The hallmarks of aging. Cell 153, 1194-1217 (2013).
9. Teitelbaum, S. L. Bone resorption by osteoclasts. Science 289, 1504-1508. (2000).
10. Wagner, E. F. & Karsenty, G. Genetic control of skeletal development. Curr. Opin. Genet. Dev. 11, 527-532 (2001).
11. Raisz, L. G. Pathogenesis of osteoporosis: concepts, conflicts, and prospects. J Clin. Inv. 115, 3318-3325 (2005).
12. Riggs, B. L., Khosla, S., & Melton, L. J. III. A unitary model for involutional osteoporosis: estrogen deficiency causes both type I and type II osteoporosis in postmenopausal women and contributes to bone loss in aging men. J Bone Miner. Res. 13, 763-773 (1998).
13. Khosla, S. & Riggs, B. L. Pathophysiology of age-related bone loss and osteoporosis. Endocrinol. Metab. Clin. North Am. 34, 1015-1030 (2005).
14. Sun, L. et al. FSH directly regulates bone mass. Cell 125, 247-260 (2006).
15. Ghosh, S. & Karin, M. Missing pieces in the NF-κB puzzle. Cell 109 (Suppl.), S81-S96 (2002).
16. Boyce, B. F., Yao, Z. & Xing, L. Functions of nuclear factor kappaB in bone. Ann. N Y Acad. Sci. 1192, 367-375 (2010).
17. Jimi, E. & Ghosh, S. Role of nuclear factor-KB in the immune system and bone. Immunol. Rev. 208, 80-87 (2005).
18. Krum, S. A., Chang, J., Miranda-Carboni, G. & Wang, C. Y. Novel functions for NF-κB: inhibition of bone formation. Nat. Rev. Rheumatol. 6, 607-611 (2010).
19. Almeida, M., Han, L., Ambrogini, E., Bartell, S. M. & Manolagas, S. C. Oxidative stress stimulates apoptosis and activates NF-kappaB in osteoblastic cells via a PKCbeta/p66shc signaling cascade: counter regulation by estrogens or androgens. Mol. Endocrinol. 24, 2030-2037 (2010).
20. Jimi, E. et al. Selective inhibition of NF-κB blocks osteoclastogenesis and prevents inflammatory bone destruction in vivo. Nat. Med. 10, 617-624 (2004).
21. Chen, Q. et al. DNA damage drives accelerated bone aging via an NF-κB-dependent mechanism. J Bone Miner. Res. 5, 1214-1228 (2013).
22. Chang, J et al. Inhibition of osteoblastic bone formation by nuclear factor-kappaB. Nat Med. 15, 682-689 (2009).
23. Anastas, J. N. & Moon, R. T. WNT signalling pathways as therapeutic targets in cancer. Nat Rev Cancer 13, 11-26 (2013).
24. MacDonald, B. T., Tamai, K. & He, X. Wnt/β-catenin signaling: components, mechanisms, and diseases. Dev. Cell 17, 9-26 (2009).
25. Regard, J. B., Zhong, Z., Williams, B. O. & Yang, Y. Wnt signaling in bone development and disease: making stronger bone with Wnts. Cold Spring Harb Perspect Biol. 4, 1-12 (2012).
26. Baron, R. & Kneissel, M. WNT signaling in bone homeostasis and disease: from human mutations to treatments. Nat Med. 19, 179-192 (2013).
27. Veeman, M. T., Axelrod, J. D. & Moon, R. T. A second canon. Functions and mechanisms of beta-catenin-independent Wnt signaling. Dev. Cell 5, 367-377 (2003).
28. Seifert, J. R. & Mlodzik, M. Frizzled/PCP signalling: a conserved mechanism regulating cell polarity and directed motility. Nat Rev Genet. 8, 126-138 (2007).
29. McNeill, H. & Woodgett, J. R. When pathways collide: collaboration and connivance among signalling proteins in development. Nat Rev Mol Cell Biol. 11, 404-413 (2010).
30. Lako, M. et al. Isolation, characterisation and embryonic expression of WNT11, a gene which maps to 11q13.5 and has possible roles in the development of skeleton, kidney and lung. Gene 219, 101-10 (1998).
31. Qiu, W., Chen, L. & Kassem, M. Activation of non-canonical Wnt/JNK pathway by Wnt3a is associated with differentiation fate determination of human bone marrow stromal (mesenchymal) stem cells. Biochem Biophys Res Commun. 413, 98-104 (2011).
32. Maeda, K. et al. Wnt5a-Ror2 signaling between osteoblast-lineage cells and osteoclast precursors enhances osteoclastogenesis. Nat. Med. 18, 405-12 (2012).
33. Chang, J. et al. Noncanonical Wnt-4 signaling enhances bone regeneration of mesenchymal stem cells in craniofacial defects through activation of p38 MAPK. J Biol. Chem. 282, 30938-30948 (2007).
34. Krebsbach, P. H. et al. Transgenic expression of COL1A1-chloramphenicol acetyltransferase fusion genes in bone: differential utilization of promoter elements in vivo and in cultured cells. Mol. Cell. Biol. 13, 5168-5174 (1993).
35. Liu, F. et al. Expression and activity of osteoblast-targeted Cre recombinase transgenes in murine skeletal tissues. Int J Dev Biol. 48, 645-653 (2004).
36. Khosla, S., Westendorf, J. J. & Oursler, M. J. Building bone to reverse osteoporosis and repair fractures. J Clin. Inv. 118, 421-428 (2008).
37. Halleen, J. M. et al. Tartrate-resistant acid phosphatase 5b: a novel serum marker of bone resorption. J Bone Miner. Res. 15, 1337-1345 (2000).
38. Pacifici, R. et al. Effect of surgical menopause and estrogen replacement on cytokine release from human blood mononuclear cells. Proc Natl Acad Sci USA. 88, 5134-5138 (1991).
39. Jilka, R. L. et al. Increased osteoclast development after estrogen loss: mediation by interleukin-6 Science 257, 88-91 (1992).
40. Hayward, M. D. et al. An extensive phenotypic characterization of the hTNFα transgenic mice. BMC Physiology 7, 13 (2007).
41. Guo, R. et al. Ubiquitin ligase Smurf1 mediates tumor necrosis factor-induced systemic bone loss by promoting proteasomal degradation of bone morphogenetic signaling proteins. J Biol. Chem. 283, 23084-23092 (2008).
42. Yao, Z., Xing, L. & Boyce, B. F. NF-kappaB p100 limits TNF-induced bone resorption in mice by a TRAF3-dependent mechanism. J Clin. Inv. 119, 3024-3034 (2009).
43. Lam, J. et al. TNF-alpha induces osteoclastogenesis by direct stimulation of macrophages exposed to permissive levels of RANK ligand. J Clin Invest. 106, 1481-1488 (2000).
44. Pandey, A. C. et al. MicroRNA profiling reveals age-dependent differential expression of nuclear factor KB and mitogen-activated protein kinase in adipose and bone marrow-derived human mesenchymal stem cells. Stem Cell Res. Ther. 2, 49 (2011).
45. Garnero, P., Sornay-Rendu, E., Chapuy, M. C. & Delmas, P. D. Increased bone turnover in late postmenopausal women is a major determinant of osteoporosis. J Bone Miner Res. 11, 337-349 (1996).
46. Ginaldi, L., Di Benedetto, M. C. & De Martinis, M. Osteoporosis, inflammation and ageing. Immun Ageing 2, 14 (2005).
47. Lomaga, M. A. et al. (1999). TRAF6 deficiency results in osteopetrosisa and defective interleukin-1, CD40, and LPS signaling. Genes Dev. 13, 1015-1024.
48. Bai, S., Zha, J., Zhao, H., Ross, F. P. & Teitelbaum, S. L. Tumor necrosis factor receptor-associated factor 6 is an intranuclear transcriptional coactivator in osteoclasts. J Biol Chem. 283, 30861-30867 (2008).
49. Mizukami, J. et al. Receptor activator of NF-kappaB ligand (RANKL) activates TAK1 mitogen-activated protein kinase kinase kinase through a signaling complex containing RANK, TAB2, and TRAF6. Mol. Cell. Biol. 22, 992-1000 (2002).
50. Takada, I. et al. A histone lysine methyltransferase activated by non-canonical Wnt signalling suppresses PPAR-gamma transactivation. Nat. Cell Biol. 9, 1273-1285 (2007).
51. Teitelbaum, S. L. & Ross, F. P. Genetic regulation of osteoclast development and function. Nat. Rev. Genet. 4, 638-649 (2003).
52. Asagiri, M. et al. Autoamplification of NFATc1 expression determines its essential role in bone homeostasis. J Exp. Med. 202, 1261-1269 (2005).
53. Holmen, S. L. et al. Essential role of β-catenin in postnatal bone acquisition. J. Biol. Chem. 280, 21162-21168 (2005).
54. Lories, R. J., Corr, M. & Lane, N. E. To Wnt or not to Wnt: the bone and joint health dilemma. Nat. Rev. Rheumatol. 9, 328-339 (2013).
55. Winkel, A, et al. Wnt-ligand-dependent interaction of TAK1 (TGF-beta-activated kinase-1) with the receptor tyrosine kinase Ror2 modulates canonical Wnt-signalling. Cell Signal. 20, 2134-2144 (2008).
56. Li, M. et al. TAB2 scaffolds TAK1 and NLK in repressing canonical Wnt signaling. J Biol. Chem. 285, 13397-13404 (2010).
59. Sugimura, R. et al. Noncanonical Wnt signaling maintains hematopoietic stem cells in the niche. Cell 150, 351-365 (2012).
60. Heinonen, K. M., Vanegas, J. R., Lew, D., Krosl, J. & Perreault, C. Wnt4 enhances murine hematopoietic progenitor cell expansion through a planar cell polarity-like pathway. PLoS One 6, e19279 (2011).
61. Fan, Z. et al. BCOR regulates mesenchymal stem cell function by epigenetic mechanisms. Nat. Cell Biol. 11, 1002-1009 (2009).
62. Redlich, K. et al. Osteoclasts are essential for TNF-alpha-mediated joint destruction. J Clin. Inv. 110, 1419-27 (2002).
63. Thwin, M. M. et al. Effect of phospholipase A2 inhibitory peptide on inflammatory arthritis in a TNF transgenic mouse model: a time-course ultrastructural study. Arthritis Res Ther. 6, 282-294 (2004).
64. Li, J. et al. LATS2 suppresses oncogenic Wnt signaling by disrupting β-catenin/BCL9 interaction. Cell Rep. 5, 1650-1663 (2013).

### Example 2

### Studies on Reversal of Bone Loss by rWnt4 Proteins

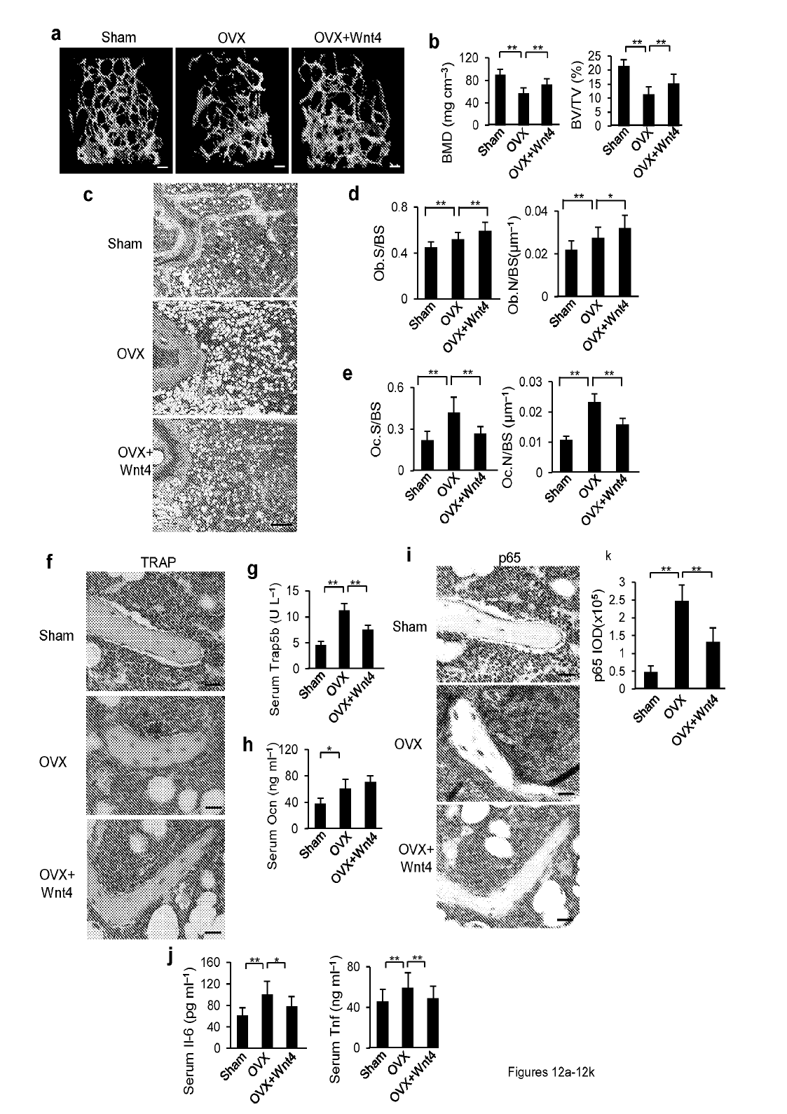

To further evaluate the therapeutic potential of rWnt4 proteins, we examined whether rWnt4 could reversed established bone loss in mice induced by ovariectomy (OVX). We first performed OVX on 3 month-old mice and waited for one month to establish bone loss. Mice were then administrated with rWnt4 or the vehicle control for one month. The therapeutic effect of rWnt4 on osteoporotic bone loss was evaluated 2 months after OVX. CT analysis revealed that rWnt4 significantly reversed OVX-induced reduction in trabecular BMD and BV/TV (FIGS. 12a,b). Histological staining confirmed that rWnt4 significantly reduced trabecular bone loss induced by OVX (FIG. 12c). Histomorphometric analysis also showed that rWnt4 significantly increased osteoblast number and surface counts induced by OVX (FIG. 12d). In contrast, rWnt4 significantly inhibited osteoclast formation induced by OVX (FIGS. 12e,f). Consistently, rWnt4 significantly reduced serum TRAP5b levels induced by OVX (FIG. 9g) while it modestly increased serum OCN levels (FIG. 12h). Immunostaining revealed that rWnt4 potently inhibited NF-kB activity in osteoclasts and adjacent bone marrow cells (FIGS. 12i,j). Again, rWnt4 proteins also significantly reduced serum levels of IL-6 and TNF induced by OVX (FIG. 12k).

Those skilled in the art will know, or be able to ascertain, using no more than routine experimentation, many equivalents to the specific embodiments of the invention described herein. These and all other equivalents are intended to be encompassed by the following claims.

1. A method for treating, ameliorating, or preventing a bone condition, comprising administering to a subject in need thereof a biologically active agent in an effective amount for promoting bone formation and inhibiting bone resorption independent of Wnt/b-catenin signaling to promote bone repair or regeneration in the subject,
wherein the biologically active agent is a Wnt4 protein.

2. (canceled)

3. The method of claim 1, wherein the Wnt4 protein is included in a pharmaceutical composition.

4. The method of claim 3, wherein the pharmaceutical composition is in a formulation for systemic delivery.

5. The method of claim 3, wherein the pharmaceutical composition is in a formulation for local delivery.

6. The method of claim 1, wherein administering comprising administering to the subject a gene construct encoding the Wnt4 protein.

7. The method of claim 1, wherein administering comprising transfecting a cell of the subject a gene construct encoding the Wnt4 protein.

8. The method of claim 1, wherein administering comprises administering to the subject an mRNA encoding the Wnt4 protein.

9. The method of claim 1, wherein the subject is a human being.

10. The method of claim 9, wherein the bone condition is selected from the group consisting of osteoporosis, inflammatory bone diseases, periodontal diseases, and chronic diseases-associated bone loss.

11. The method of claim 9, wherein the bone condition is arthritis.

12. A composition for bone regeneration in a subject, comprising a biologically active agent in an effective amount for promoting bone formation and inhibiting bone resorption in the animal independent of Wnt/b-catenin signaling,
wherein the biologically active agent is a Wnt4 protein.

13. (canceled)

14. The composition of claim 12, wherein the Wnt4 protein is included in a pharmaceutical composition.

15. The composition of claim 14, wherein the pharmaceutical composition is in a formulation for systemic delivery.

16. The composition of claim 14, wherein the pharmaceutical composition is in a formulation for local delivery.

17. The composition of claim 12, wherein the subject is a human being.

18. The composition of claim 17, wherein the bone condition is selected from the group consisting of osteoporosis, inflammatory bone diseases, periodontal diseases, and chronic diseases-associated bone loss.

19. The composition of claim 12, wherein the bone condition is arthritis.

20. The composition of claim 15, comprising a gene construct encoding the Wnt4 protein.

21. The composition of claim 15, comprising an mRNA encoding the Wnt4 protein.

22. A method of fabricating a composition for treating, ameliorating, or preventing a bone condition, comprising providing a biologically active agent in an effective amount for promoting bone formation and inhibiting bone resorption independent of Wnt/b-catenin signaling to promote bone repair or regeneration in a subject,
wherein the biologically active agent is a Wnt4 protein.

23. (canceled)

24. The method of claim 22, wherein the Wnt4 protein is included in a pharmaceutical composition.

25. The method of claim 24, wherein the pharmaceutical composition is in a formulation for systemic delivery or in a formulation for local delivery.

26-28. (canceled)
